(19) United States
(12) Patent Application Publication
(10) Pub. No.: US 2024/0269136 A1
(43) Pub. Date: Aug. 15, 2024

(54) EGFR INHIBITOR AND PERK ACTIVATOR IN COMBINATION THERAPY AND THEIR USE FOR TREATING CANCER

(71) Applicant: Genentech, Inc., South San Francisco, CA (US)

(72) Inventors: Scott Andrew FOSTER, San Mateo, CA (US); Xin YE, Burlingame, CA (US)

(73) Assignee: Genentech, Inc., South San Francisco, CA (US)

(21) Appl. No.: 18/540,310

(22) Filed: Dec. 14, 2023

Related U.S. Application Data
(63) Continuation of application No. PCT/US2022/033168, filed on Jun. 13, 2022.
(60) Provisional application No. 63/210,925, filed on Jun. 15, 2021.

(51) Int. Cl. A61K 31/505; A61K 31/357; A61K 31/4709; A61K 31/506; A61K 31/517; A61K 31/519; A61K 31/5377; A61K 31/55

(52) U.S. Cl. CPC A61K 31/505; A61K 31/357; A61K 31/4709; A61K 31/506; A61K 31/517; A61K 31/519; A61K 31/5377; A61K 31/55

(57) ABSTRACT

Provided herein are combinations comprising: (i) one or more EGFR inhibitors; and (ii) one or more PERK activators, such as the specific EGFR inhibitors and PERK activators described herein. Also provided herein are methods of treating cancer in a subject in need thereof, comprising administering to the subject an effective amount of such combinations.

### CROSS-REFERENCE TO RELATED APPLICATIONS

This application is a Continuation of International Application No. PCT/US2022/033168, filed on Jun. 13, 2022, which claims the benefit of priority to U.S. Provisional Application No. 63/210,925, filed on Jun. 15, 2021, the contents of each of which are incorporated by reference herein in their entirety.

### BACKGROUND

Epidermal growth factor receptor (EGFR) is a trans-membrane glycoprotein which belongs to a family of structurally related receptor tyrosine kinases. EGFR feeds into the PI3K/AKT/mTOR and MAPK pathways at the cell surface level. EGFR is believed to be important in multiple signal-transduction pathways and appears to play a critical role in both tumorigenesis and tumor growth. EGFR and its ligands are overexpressed or involved in autocrine growth loops in a number of tumor types.

The EGFR family comprises four closely related receptors (HER1/EGFR, HER2, HER3 and HER4) involved in cellular responses such as differentiation and proliferation. Mutation of the EGFR kinase, or overexpression of its ligands such as TGF-alpha and EGF, is frequently associated with many cancers, including breast, lung, colorectal, ovarian, renal cell, bladder, head and neck cancers, glioblastomas, and astrocytomas, and is believed to contribute to the malignant growth of these tumors. A specific deletion-mutation in the EGFR gene (EGFRvIII) has also been found to increase cellular tumorigenicity. Activation of EGFR stimulated signaling pathways promote multiple processes that are potentially cancer-promoting, e.g. proliferation, angiogenesis, cell motility and invasion, decreased apoptosis and induction of drug resistance. Increased HER1/EGFR expression is frequently linked to advanced disease, metastases and poor prognosis. For example, in NSCLC and gastric cancer, increased HER1/EGFR expression has been shown to correlate with a high metastatic rate, poor tumor differentiation and increased tumor proliferation. Targeted therapies such as those that inhibit the EGFR signaling pathway has been extensively evaluated in patients with EGFR-mutant NSCLC. (Pao et al., Nat Rev Cancer 2010; 10:760-774).

First-line therapies incorporating first generation EGFR-TKIs such as gefitinib (IRESSA) and erlotinib (TARCEVA) have shown to be effective in advanced NSCLC harboring recurrent somatic activating mutations occurring in the exons encoding the kinase domain of EGFR, i.e., small multi-nucleotide in-frame deletions in exon 19 (ex19del) and a point mutation in exon 21 leading to substitution of leucine for arginine at position 858 (L858R) (Lynch et al., N Eng J Med 2004; 350:2129-39; Paez et al., Science 2004; 304: 1497-500; Pao et al., PNAS 2004; 101: 13306-11). These somatic mutations in EGFR lead to constitutive activation of EGFR signaling and oncogenic transformation (Ji et al., Cancer Cell 2006; 9:485-95).

Subsequent generations (e.g., second, third, and forth generation) of EGFR TKIs have been developed to inhibit various mutant forms of EGFR, as well as wild-type EGFR. It has been shown that while a reasonable number of patients with EGFR-mutant NSCLC respond to EGFR-TKI therapy initially, most patients who respond to therapy ultimately develop disease progression after about 9-14 months of treatment due to acquired resistance to the EGFR-TKI (Maemondo et al., N Eng J Med 2010; 362:2380-8). In approximately 60% of these patients treated with first generation EGFR-TKIs, the mechanism of acquired resistance is the development of an additional EGFR mutation, T790M (Yu et al., Clin Cancer Res 2013; 19:2240-7). The T790M substitution leads to an enhanced affinity for ATP, reducing the ability of ATP-competitive reversible EGFR tyrosine kinase inhibitors such as gefitinib and erlotinib to bind to the tyrosine kinase domain of EGFR (Yun et al., PNAS 2008; 105:2070-5). Other mutations leading to EGFR-TKI acquired resistance include MET amplification, HER2 amplification, epithelial-mesenchymal transition (EMT), and transformation of NSCLC to small-cell histology (Takezawa et al., Cancer Discov. 2012; 2(10):922-933; Uramoto et al., Lung Cancer 2011; 73(3):361-365).

Second-generation EGFR-TKIs are irreversible EGFR-TKIs, which have more potent EGFR inhibition than gefitinib and erlotinib. These second-generation EGFR-TKIs, however, are also prone to acquired resistance and have limited clinical utility due to a lack of mutant selectivity which results in profound on-target toxicity. Third generation EGFR TKI are irreversible EGFR-TKIs including osimertinib (TAGRISSO), rociletinib (CO-1686), olmutinib (OLITA; BI 1482694; HM61713), naquotinib (ASP8273), nazartinib (EGF816), and mavelertinib (PF-06747775). Third generation EGFR TKIs have emerged to target both EGFR-mutant and EGFR-mutant T790M-positive NSCLC (Cross et al., Cancer Discov. 2014; 4(9): 1046-61; Walter et al., Cancer Discov. 2013; 3(12):404-15; Park et al., ASCO Meet Abstr. 2015; 33(15)8084; Wang et al., J. Hemat. & Oncol. 2016; 9:34). Treatment with either osimertinib or rociletinib has resulted in a response rate of >50% in EGFR-mutant EGFR-T790M NSCLC patients who have developed resistance to gefitinib or erlotinib (Janne et al., N Eng J Med 2015; 372: 1689-99; Sequist et al., N Eng J Med 2015; 372: 1700-9). The third-generation EGFR-TKIs, however, are also prone to eventual ineffectiveness through acquired resistance, with mechanisms of resistance far more diverse than first generation inhibitors and secondary site EGFR mutations only occurring in a small fraction of patients (Leonetti et al. Br J Cancer (2019)). The most common on-target mutation leading to resistance includes C797 mutations C797S and C797G (Ercan et al., Clin Can Res 2015 21(17)3913-3923). Cysteine 797 is the site of covalent binding for second and third-generation inhibitors, including both rociletinib and osimertinib (Zhou et al., Nature 2009; 462: 1070-4). Fourth generation EGFR TKI have emerged to target the C797S and C797G mutation. For example, EAI045 is a fourth generation EGFR TKI which inhibits L858R/T790M EGFR mutant NSCLC, as well as C797S and C797G EGFR mutant NSCLC (Wang et al. Cancer Lett. 2017, 385:51-54).

Integrated stress response (ISR) is a cellular stress response conserved in eukaryotic cells that downregulates protein synthesis and upregulates specific genes in response to internal or environmental stresses. Specific stress signals can result in activation of one of a family of 4 protein kinases, known as EIF-2 kinases, depending upon the specific stress. EIF2alpha kinases phosphorylate the a subunit of a protein complex called translation initiation factor 2 (eIF2), resulting in activation of activating transcription factor 4 (ATF4), which will further affect ATF4 gene expression. There are four known mammalian protein kinases that phosphorylate eIF2α, including PKR-like ER kinase (PERK, EIF2AK3), heme-regulated eIF2α kinase (HRI, EIF2AK1), general control non-depressible 2 (GCN2, EIF2AK4) and double stranded RNA dependent protein kinase (PKR, EIF2AK2). PERK (encoded in humans by the gene EIF2AK3) responds mainly to endoplasmic reticulum stress and has two modes of activation, including activation by aggregation of unfolded proteins in the endoplasmic reticulum and activation in response to activity of the proto-oncogene MYC.

Lung cancer is the most common and deadly cancer worldwide, with non-small cell lung cancer (NSCLC) accounting for approximately 85% of lung cancer cases. In Western countries, 10-15% of NSCLC patients express epidermal growth factor receptor (EGFR) mutations in their tumors, and Asian counties have reported rates as high as 30-40%. The predominant oncogenic EGFR mutations (L858R and ex19del) account for about 85% of EGFR mutant NSCLC. Currently, EGFR-mutant patients are treated with an EGFR tyrosine kinase inhibitor (TKI) as first line therapy. However, most patients develop acquired resistance to first and second-generation inhibitors, generally within 10 to 14 months. In 50-60% of NSCLC patients harboring a primary EGFR mutation treated with first-generation reversible EGFR tyrosine kinase, a secondary “gatekeeper” T790M mutation develops. Third-generation EGFR TKIs have been developed to try to overcome this mechanism of resistance. However, resistance to third-generation inhibitors also develops, with patients developing a range of resistance mechanisms over time. Therefore, there exists a need for improved therapeutic options for treating EGFR-related diseases and conditions such as EGFR-mutant cancer.

### SUMMARY

Provided here are compositions, methods and kits comprising an EGFR inhibitor and a PERK activator.

Various embodiments are contemplated herein. For example in Embodiment 1, provided is a method of treating cancer in an individual in need thereof, comprising administering to the individual a therapeutically effective amount of a combination of (i) a first therapy comprising an EGFR inhibitor, and (ii) a second therapy comprising a PERK activator.

Embodiment 2: The method of Embodiment 1, wherein the PERK activator is selected from the group consisting of 1-(4-((4-((5-cyclopropyl-1H-pyrazol-3-yl)amino)pyrimidin-2-yl)amino)phenyl)-3-(4-(methylthio)phenyl)urea (IPA), 4-[(E)-[(4,6-diphenylpyrimidin-2-yl)-methyl-hydrazono]methyl]benzene-1,2-diol (MK-28), 3-(2,3-dihydrobenzo[b][1,4]dioxin-6-yl)-5,7-dihydroxy-4H-chromen-4-one (DHBDC), 6-bromo-3-[3-(4-bromophenyl)-2-[3-(diethylamino)propanoyl]-3,4-dihydropyrazol-5-yl]-4-phenyl-1H-quinolin-2-one (CCT-020312), and pharmaceutically acceptable salts thereof

Embodiment 3: The method of Embodiments 1 or 2, wherein the EGFR inhibitor is a small molecule EGFR tyrosine kinase inhibitor (EGFR TKI).

Embodiment 4: The method of any one of Embodiments 1 to 3, wherein the EGFR inhibitor is a first generation EGFR TKI, a second generation EGFR TKI, a third generation EGFR TKI, or a fourth generation EGFR TKI.

Embodiment 5: The method of any one of Embodiments 1 to 4, wherein the EGFR inhibitor binds reversibly to the EGFR tyrosine kinase domain.

Embodiment 6: The method of any one of Embodiments 1 to 4, wherein the EGFR inhibitor binds irreversibly to the EGFR tyrosine kinase domain.

Embodiment 7: The method of any one of Embodiments 1 to 6, wherein the EGFR inhibitor is an inhibitor of wild-type EGFR, and/or a mutant form of EGFR, wherein the mutant form of EGFR is exon20 insertion, S768I, ex19del, L858R, L861Q, T790M, C797S, C797G, T854A, D761Y, L747S, G719X, L861X, V8343I, V769M, A871E, or any combination thereof.

Embodiment 8: The method of any one of Embodiments 1 to 3, wherein the EGFR inhibitor is selected from the group consisting of rac-(E)-N-[4-(3-chloro-4-fluoro-anilino)-7-[rac-(3S)-tetrahydrofuran-3-yl]oxy-quinazolin-6-yl]-4-(dimethylamino)but-2-enamide (afatinib), (E)-N-[4-(3-chloro-4-fluoro-anilino)-7-methoxy-quinazolin-6-yl]-4-(1-piperidyl)but-2-enamide (dacomitinib), N-(3-ethynylphenyl)-6,7-bis(2-methoxyethoxy)quinazolin-4-amine (erlotinib), N-(3-chloro-4-fluoro-phenyl)-7-methoxy-6-(3-morpholinopropoxy)quinazolin-4-amine (gefitinib), N-[7-chloro-1-[rac-(3R)-1-[rac-(E)-4-(dimethylamino)but-2-enoyl]azepan-3-yl]benzimidazol-2-yl]-2-methyl-pyridine-4-carboxamide (nazartinib), N-[2-[2-(dimethylamino)ethyl-methyl-amino]-4-methoxy-5-[[4-(1-methylindol-3-yl)pyrimidin-2-yl]amino]phenyl]prop-2-enamide (osimertinib), 1-[4-[4-(3,4-dichloro-2-fluoro-anilino)-7-methoxy-quinazolin-6-yl]oxy-1-piperidyl]prop-2-en-1-one (poziotinib), N-[3-[[2-[4-(4-acetylpiperazin-1-yl)-2-methoxy-anilino]-5-(trifluoromethyl)pyrimidin-4-yl]amino]phenyl]prop-2-enamide (rociletinib), N-[rac-(8S)-4-amino-6-methyl-5-(3-quinolyl)-8,9-dihydropyrimido[5,4-b]indolizin-8-yl]prop-2-enamide (TAS-6417), and N-[3-[5-chloro-2-[2-methoxy-4-(4-methylpiperazin-1-yl)anilino]pyrimidin-4-yl]oxyphenyl]prop-2-enamide (WZ-4002), and pharmaceutically acceptable salts thereof.

Embodiment 9: The method of any one of Embodiments 1 to 8, wherein the cancer is squamous cell cancer, lung cancer, cancer of the peritoneum, hepatocellular cancer, gastric or stomach cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, B-cell lymphoma, chronic lymphocytic leukemia (CLL), acute lymphoblastic leukemia (ALL), Hairy cell leukemia, chronic myeloblastic leukemia, post-transplant lymphoproliferative disorder (PTLD), abnormal vascular proliferation associated with phakomatoses, edema, or Meigs' syndrome.

Embodiment 10: The method of Embodiment 9, wherein the lung cancer is small-cell lung cancer, non-small cell lung cancer (NSCLC), adenocarcinoma of the lung, or squamous carcinoma of the lung.

Embodiment 11: The method of any one of Embodiments 1 to 8, wherein the cancer is an EGFR-mutant cancer selected from the group consisting of bladder cancer, glioblastoma, head and neck cancer, breast cancer, cervical cancer, uterine cancer, colorectal cancer, gastroesophageal cancer, non-small cell lung carcinoma (NSCLC), prostate cancer, ovarian cancer, pancreatic cancer, renal cell carcinoma, squamous cell carcinoma, and thyroid cancer.

Embodiment 12: The method of any one of Embodiments 1 to 8, wherein the individual is diagnosed with NSCLC, prostate cancer, breast cancer, colon cancer, or pancreatic cancer.

Embodiment 13: The method of Embodiment 12, wherein the NSCLC is metastatic NSCLC or locally advanced NSCLC.

Embodiment 14: The method of Embodiment 12, wherein the individual is diagnosed with EGFR exon 19 deletions positive NSCLC or exon 21 L858R mutations positive NSCLC.

Embodiment 15: The method of Embodiment 12, wherein the individual is diagnosed with EGFR T790M mutation positive NSCLC.

Embodiment 16: The method of any one of Embodiments 1 to 15, wherein the therapeutically effective amount of the second therapy is sufficient to increase efficacy of the EGFR inhibitor.

Embodiment 17: The method of any one of Embodiments 1 to 16, wherein the first and second therapies are administered sequentially.

Embodiment 18: The method of any one of Embodiments 1 to 16, wherein the first and second therapies are administered simultaneously.

Embodiment 19: The method of any one of Embodiments 1 to 18, wherein the first and second therapies are contained in the same pharmaceutical composition.

Embodiment 20: The method of any one of Embodiments 1 to 18, wherein the first and second therapies are contained in separate pharmaceutical composition.

Embodiment 21: The method of any one of Embodiments 1 to 20, wherein the method comprises a third anti-cancer therapy.

Embodiment 22: The method of Embodiment 21, wherein the third anti-cancer therapy is chemotherapy, radiation therapy, surgery, targeted therapy, immunotherapy, hormone therapy, or stem cell or bone marrow transplant.

Embodiment 23: A kit comprising: (a) a first therapy comprising an EGFR inhibitor; (b) a second therapy comprising a PERK activator; and (c) instructions for using the kit in treating cancer in an individual.

Embodiment 24: The kit of Embodiment 23, wherein the PERK activator is selected from the group consisting of 1-(4-((4-((5-cyclopropyl-1H-pyrazol-3-yl)amino)pyrimidin-2-yl)amino)phenyl)-3-(4-(methylthio)phenyl)urea (IPA), 4-[(E)-[(4,6-diphenylpyrimidin-2-yl)-methyl-hydrazono]methyl]benzene-1,2-diol (MK-28), 3-(2,3-dihydrobenzo[b][1,4]dioxin-6-yl)-5,7-dihydroxy-4H-chromen-4-one (DHBDC), 6-bromo-3-[3-(4-bromophenyl)-2-[3-(diethylamino)propanoyl]-3,4-dihydropyrazol-5-yl]-4-phenyl-1H-quinolin-2-one (CCT-020312), and pharmaceutically acceptable salts thereof.

Embodiment 25: A pharmaceutical composition comprising: (a) a first therapy comprising an EGFR inhibitor; (b) a second therapy comprising a PERK activator; and (c) a pharmaceutically acceptable carrier.

Embodiment 26: The pharmaceutical composition of Embodiment 25, wherein the PERK activator is selected from the group consisting of 1-(4-((4-((5-cyclopropyl-1H-pyrazol-3-yl)amino)pyrimidin-2-yl)amino)phenyl)-3-(4-(methylthio)phenyl)urea (IPA), 4-[(E)-[(4,6-diphenylpyrimidin-2-yl)-methyl-hydrazono]methyl]benzene-1,2-diol (MK-28), 3-(2,3-dihydrobenzo[b][1,4]dioxin-6-yl)-5,7-dihydroxy-4H-chromen-4-one (DHBDC), 6-bromo-3-[3-(4-bromophenyl)-2-[3-(diethylamino)propanoyl]-3,4-dihydropyrazol-5-yl]-4-phenyl-1H-quinolin-2-one (CCT-020312), and pharmaceutically acceptable salts thereof.

Embodiment 27: A composition comprising a unit dosage form of a first therapy comprising an EGFR inhibitor and a unit dosage form of a second therapy comprising a PERK activator.

Embodiment 28: The composition of Embodiment 27, wherein the PERK activator is selected from the group consisting of 1-(4-((4-((5-cyclopropyl-1H-pyrazol-3-yl)amino)pyrimidin-2-yl)amino)phenyl)-3-(4-(methylthio)phenyl)urea (IPA), 4-[(E)-[(4,6-diphenylpyrimidin-2-yl)-methyl-hydrazono]methyl]benzene-1,2-diol (MK-28), 3-(2,3-dihydrobenzo[b][1,4]dioxin-6-yl)-5,7-dihydroxy-4H-chromen-4-one (DHBDC), 6-bromo-3-[3-(4-bromophenyl)-2-[3-(diethylamino)propanoyl]-3,4-dihydropyrazol-5-yl]-4-phenyl-1H-quinolin-2-one (CCT-020312), and pharmaceutically acceptable salts thereof.

Embodiment 29: A method of treating cancer in an individual in need thereof, comprising administering to the individual a therapeutically effective amount of a combination of
(i) a first therapy comprising an EGFR inhibitor selected from the group consisting of rac-(E)-N-[4-(3-chloro-4-fluoro-anilino)-7-[rac-(3S)-tetrahydrofuran-3-yl]oxy-quinazolin-6-yl]-4-(dimethylamino)but-2-enamide (afatinib), (E)-N-[4-(3-chloro-4-fluoro-anilino)-7-methoxy-quinazolin-6-yl]-4-(1-piperidyl)but-2-enamide (dacomitinib), N-(3-ethynylphenyl)-6,7-bis(2-methoxyethoxy)quinazolin-4-amine (erlotinib), N-(3-chloro-4-fluoro-phenyl)-7-methoxy-6-(3-morpholinopropoxy)quinazolin-4-amine (gefitinib), N-[7-chloro-1-[rac-(3R)-1-[rac-(E)-4-(dimethylamino)but-2-enoyl]azepan-3-yl]benzimidazol-2-yl]-2-methyl-pyridine-4-carboxamide (nazartinib), N-[2-[2-(dimethylamino)ethyl-methyl-amino]-4-methoxy-5-[[4-(1-methylindol-3-yl)pyrimidin-2-yl]amino]phenyl]prop-2-enamide (osimertinib), 1-[4-[4-(3,4-dichloro-2-fluoro-anilino)-7-methoxy-quinazolin-6-yl]oxy-1-piperidyl]prop-2-en-1-one (poziotinib), N-[3-[[2-[4-(4-acetylpiperazin-1-yl)-2-methoxy-anilino]-5-(trifluoromethyl)pyrimidin-4-yl]amino]phenyl]prop-2-enamide (rociletinib), N-[rac-(8S)-4-amino-6-methyl-5-(3-quinolyl)-8,9-dihydropyrimido[5,4-b]indolizin-8-yl]prop-2-enamide (TAS-6417), and N-[3-[5-chloro-2-[2-methoxy-4-(4-methylpiperazin-1-yl)anilino]pyrimidin-4-yl]oxyphenyl]prop-2-enamide (WZ-4002), and pharmaceutically acceptable salts thereof, and
(ii) a second therapy comprising a PERK activator selected from the group consisting of 1-(4-((4-((5-cyclopropyl-1H-pyrazol-3-yl)amino)pyrimidin-2-yl)amino)phenyl)-3-(4-(methylthio)phenyl)urea (IPA), 4-[(E)-[(4,6-diphenylpyrimidin-2-yl)-methyl-hydrazono]methyl]benzene-1,2-diol (MK-28), 3-(2,3-dihydrobenzo[b][1,4]dioxin-6-yl)-5,7-dihydroxy-4H-chromen-4-one (DHBDC), 6-bromo-3-[3-(4-bromophenyl)-2-[3-(diethylamino)propanoyl]-3,4-dihydropyrazol-5-yl]-4-phenyl-1H-quinolin-2-one (CCT-020312), and pharmaceutically acceptable salts thereof.

Embodiment 30: The method of Embodiment 29, wherein the cancer is an EGFR-mutant cancer selected from the group consisting of bladder cancer, glioblastoma, head and neck cancer, breast cancer, cervical cancer, uterine cancer, colorectal cancer, gastroesophageal cancer, non-small cell lung carcinoma (NSCLC), prostate cancer, ovarian cancer, pancreatic cancer, renal cell carcinoma, squamous cell carcinoma, and thyroid cancer.

Embodiment 31: The method of Embodiments 29 or 30, wherein the method comprises a third anti-cancer therapy, wherein the third anti-cancer therapy is chemotherapy, radiation therapy, surgery, targeted therapy, immunotherapy, hormone therapy, or stem cell or bone marrow transplant.

Embodiment 32: Use of an EGFR inhibitor in the manufacture of a medicament for use in the treatment of a subject suffering from cancer in combination with a PERK activator.

Embodiment 33: Use of a PERK activator in the manufacture of a medicament for use in the treatment of a subject suffering from cancer in combination with an EGFR inhibitor.

Embodiment 34: Use of a combination of an EGFR inhibitor and a PERK activator in the manufacture of a medicament for use in the treatment of a subject suffering from cancer.

Embodiment 35: The use of any one of Embodiments 32-34, wherein the EGFR inhibitor is selected from the group consisting of rac-(E)-N-[4-(3-chloro-4-fluoro-anilino)-7-[rac-(3S)-tetrahydrofuran-3-yl]oxy-quinazolin-6-yl]-4-(dimethylamino)but-2-enamide (afatinib), (E)-N-[4-(3-chloro-4-fluoro-anilino)-7-methoxy-quinazolin-6-yl]-4-(1-piperidyl)but-2-enamide (dacomitinib), N-(3-ethynylphenyl)-6,7-bis(2-methoxyethoxy)quinazolin-4-amine (erlotinib), N-(3-chloro-4-fluoro-phenyl)-7-methoxy-6-(3-morpholinopropoxy)quinazolin-4-amine (gefitinib), N-[7-chloro-1-[rac-(3R)-1-[rac-(E)-4-(dimethylamino)but-2-enoyl]azepan-3-yl]benzimidazol-2-yl]-2-methyl-pyridine-4-carboxamide (nazartinib), N-[2-[2-(dimethylamino)ethyl-methyl-amino]-4-methoxy-5-[[4-(1-methylindol-3-yl)pyrimidin-2-yl]amino]phenyl]prop-2-enamide (osimertinib), 1-[4-[4-(3,4-dichloro-2-fluoro-anilino)-7-methoxy-quinazolin-6-yl]oxy-1-piperidyl]prop-2-en-1-one (poziotinib), N-[3-[[2-[4-(4-acetylpiperazin-1-yl)-2-methoxy-anilino]-5-(trifluoromethyl)pyrimidin-4-yl]amino]phenyl]prop-2-enamide (rociletinib), N-[rac-(8S)-4-amino-6-methyl-5-(3-quinolyl)-8,9-dihydropyrimido[5,4-b]indolizin-8-yl]prop-2-enamide (TAS-6417), and N-[3-[5-chloro-2-[2-methoxy-4-(4-methylpiperazin-1-yl)anilino]pyrimidin-4-yl]oxyphenyl]prop-2-enamide (WZ-4002), and pharmaceutically acceptable salts thereof

Embodiment 36: The use of any one of Embodiments 32-35, wherein the PERK activator is selected from the group consisting of 1-(4-((4-((5-cyclopropyl-1H-pyrazol-3-yl)amino)pyrimidin-2-yl)amino)phenyl)-3-(4-(methylthio)phenyl)urea (IPA), 4-[(E)-[(4,6-diphenylpyrimidin-2-yl)-methyl-hydrazono]methyl]benzene-1,2-diol (MK-28), 3-(2,3-dihydrobenzo[b][1,4]dioxin-6-yl)-5,7-dihydroxy-4H-chromen-4-one (DHBDC), 6-bromo-3-[3-(4-bromophenyl)-2-[3-(diethylamino)propanoyl]-3,4-dihydropyrazol-5-yl]-4-phenyl-1H-quinolin-2-one (CCT-020312), and pharmaceutically acceptable salts thereof.

### DETAILED DESCRIPTION

Unless specifically defined otherwise, all technical and scientific terms used herein shall be taken to have the same meaning as commonly understood by one of ordinary skill in the art (e.g., cancer biology, cancer therapy, immunology, pharmacology, protein chemistry, and biochemistry).

### Definitions

As used herein, “treatment” or “treating” is an approach for obtaining beneficial or desired clinical results. For purposes of this invention, beneficial or desired clinical results include, but are not limited to, any one or more of: alleviation of one or more symptoms, diminishment of extent of disease, preventing or delaying spread (e.g., metastasis) of disease, preventing or delaying recurrence of disease, delay or slowing of disease progression, and amelioration of the disease state. Also encompassed by “treatment” is a reduction of pathological consequence of a proliferative disease. The methods of the invention contemplate any one or more of these aspects of treatment.

A “therapeutically effective amount” refers to an amount of a compound or a combination therapy sufficient to produce a desired therapeutic outcome. As is understood in the art, a therapeutically effective amount may be in one or more doses, i.e., a single dose or multiple doses may be required to achieve the desired treatment endpoint. A therapeutically effective amount may be considered in the context of administering one or more therapeutic agents, and a single agent may be considered to be given in a therapeutically effective amount if, in conjunction with one or more other agents, a desirable or beneficial result may be or is achieved. The compounds in a combination therapy of the invention may be administered sequentially, simultaneously, or continuously using the same or different routes of administration for each compound. Thus, a therapeutically effective amount of a combination therapy includes an amount of the first therapy and an amount of the second therapy that when administered sequentially, simultaneously, or continuously produces a desired outcome. Suitable doses of any of the co-administered compounds may optionally be lowered due to the combined action (e.g., additive or synergistic effects) of the compounds.

The terms “synergy” or “synergistic” as used herein refer to any effect resulting from the use of a combination of agents where the effect is quantitatively greater than the sum of the effects resulting from the use of each agent separately. For example, a synergistic effect of a combination of therapies (e.g., a combination of a first therapy and a second therapy) may permit the use of lower dosages of one or more of the therapeutic agent(s) and/or less frequent administration of said agent(s) to an individual with a disease or disorder, e.g., cancer. The ability to utilize lower the dosage of one or more therapeutic agent and/or to administer said therapeutic agent less frequently may reduce the toxicity associated with the administration of said agent to a subject without reducing the efficacy of said therapy in the treatment of a disease or disorder. In addition, a synergistic effect may result in improved efficacy of agents in the prevention, management or treatment of a disease or disorder, e.g. cancer. Other synergistic effects of a combination of therapies may include avoiding or reducing adverse or unwanted side effects associated with the use of either therapeutic agent alone or any other benefit that is not achieved from the use of each agent separately.

The term “first therapy”, as used herein, refers to a therapy comprising one or more EGFR inhibitors. The term “second therapy”, as used herein, refers to a therapy comprising one or more PERK activators.

Administration “in combination with” another compound includes administration in the same or different composition, sequentially, simultaneously, or continuously.

The term “simultaneous administration,” as used herein, means that a first therapy and a second therapy in a combination therapy are administered to the individual at the same time or with a short time separation such as no more than about 5 minutes. When the compounds are administered simultaneously, the first and second therapies may be contained in the same composition (e.g., a composition comprising both an EGFR inhibitor and a PERK activator) or in separate compositions (e.g., an EGFR inhibitor is contained in one composition and a PERK activator is contained in another composition). In general, agents being administered in combination do not necessarily have to be administered in the same pharmaceutical composition, and may, e.g., because of different physical and chemical characteristics, be administered by different routes.

As used herein, the term “sequential administration” means that the first therapy and second therapy in a combination therapy are not administered to the individual at the same time but rather one of the therapies is administered first, and the other therapy is administered after administration of the first administered therapy with a time separation of, for example, more than about 5 minutes. Either the first therapy or the second therapy may be administered first. The first and second therapies are contained in separate compositions, which may be contained in the same or different packages or kits.

“EGFR inhibitor” as used herein, includes any compound that disrupts EGFR function within a cell. EGFR inhibitors include compounds that bind to the tyrosine kinase (TK) domain of EGFR to inhibit its enzymatic activity. For example, when an EGFR inhibitor is a compound, the EGFR inhibitor is functionally defined as any compound that inhibits the activity of EGFR and/or as a compound for use in methods to inhibit the activity of EGFR, independent of its chemical structure, i.e., not limited by its chemical structure.

“First generation EGFR TKI” as used herein, includes any compound that reversibly binds to EGFR and inhibits the binding of ATP to the TK domain.

“Second generation EGFR TKI” as used herein, includes any compound that is an irreversible EGFR TKI.

“Third generation EGFR TKI” as used herein, includes any compound that is an irreversible EGFR TKI that inhibits EGFR with mutant T790M.

“Fourth generation EGFR TKI” as used herein, includes any compound that inhibits EGFR with C797S or C797G mutations.

“PERK activator” or “PERK activating compound” as used herein, is functionally defined as a compound that simulates the kinase activity of PERK protein and/or as a compound for use in methods to stimulate the kinase activity of PERK protein independent of its chemical structure, i.e., not limited by its chemical structure. Stimulation of kinase activity of PERK protein may be determined by increased phosphorylation of its target protein eukaryotic translation initiation factor 2-alpha (EIF2A) and/or nuclear factor erythroid 2-related factor 2 (NRF2).

As used herein, “pharmaceutically acceptable” or “pharmacologically acceptable” means a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients may include materials that have met required standards of toxicological and manufacturing testing such as those included on the Inactive Ingredient Guide prepared by the U.S. Food and Drug administration.

As used herein, “cancer” refers to or describes the physiological condition in mammals that is typically characterized by unregulated cell growth. Included in this definition are benign and malignant cancers as well as dormant tumors or micrometastases. Examples of cancer include, but are not limited to, carcinoma, lymphoma, blastoma, sarcoma, and leukemia. More particular examples of such cancers include but are not limited to squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Examples of cancer may include primary tumors of any of the above types of cancer or metastatic tumors at alternative (non-original) sites derived from any of the above types of cancer.

“Tumor” as used herein, refers to abnormal growth of tissues that may or may not be cancerous.

“Wild-type EGFR” as used herein, refers to EGFR with no detectible mutation. Mutations and variant classes of EGFR include, but are not limited to, ex19del, L858R, L861Q, T790M, C797S, C797G, T854A, D761Y, L747S, G719X, L861X, V8343I, V769M, and A871E.

“EGFR-mutant cancer” as used herein, refers to cancers that are driven, in whole or in part, by EGFR-mutations. Examples of EGFR-mutant cancers include, but not limited to, bladder cancer, gliomas including glioblastoma, head and neck cancer, breast cancer, cervical cancer, uterine cancer, colon and colorectal cancer, gastroesophageal cancer, non-small cell lung carcinoma (NSCLC), prostate cancer, ovarian cancer, pancreatic cancer, renal cell carcinoma, squamous cell carcinoma, and thyroid cancer.

“Individual”, “patient” or “subject” refers to humans and other animals (such as mammals) including animals used in assays and preclinical testing including but not limited to mice, rats, monkeys, dogs, pigs and rabbits as well as domesticated swine (pigs and hogs), ruminants, equine, poultry, felines, bovines, murines, canines, and the like. “Individual”, “subject” and “patient” are used interchangeably herein. The term “adult” refers to an individual 18 years of age or older. In certain embodiments, the individual is a senior adult. e.g., 65 years or older. The term “juvenile” refers to an individual 12 to less than 18 years of age.

As used herein, the term “unit dosage form” refers to a composition that contains a predetermined dose of a compound.

Reference to “about” a value or parameter herein includes (and describes) variations that are directed to that value or parameter per se.

The singular forms “a,” “or,” and “the” include plural referents unless the context clearly dictates otherwise.

The “comprise” or variations such as “comprises” or “comprising”, will be understood to imply the inclusion of a stated element, integer or step, or group of elements, integers or steps, but not the exclusion of any other element, integer or step, or group of elements, integers or steps. Embodiments described herein include “consisting” and/or “consisting essentially of” aspects.

Provided herein are compositions, methods and kits comprising a first therapy comprising an EGFR inhibitor and a second therapy comprising a PERK activator.

### EGFR Inhibitor

In some embodiments, the first therapy comprises any agent that demonstrates EFGR inhibition activity. In some embodiments, the agent is active against wild-type EGFR, and/or various mutant forms of EGFR, including but not limited to, exon20 insertion, S768I, ex19del, L858R, L861Q, T790M, C797S, C797G, T854A, D761Y, L747S, G719X, L861X, V8343I, V769M, and A871E. In some embodiments, the EGFR inhibitor is an inhibitor of wild-type EGFR, a mutant form of EGFR, or a combination thereof. In some embodiments, the mutant form of EGFR is ex19del, L858R, L861Q, T790M, C797S, C797G, T854A, D761Y, L747S, G719X, L861X, V8343I, V769M, A871E, or any combination thereof.

In some embodiments, the first therapy comprises a first generation EGFR TKI (e.g. gefitinib, erlotinib, and icotinib). In some embodiments, the EGFR inhibitor is a second generation EGFR TKI (e.g. afatinib, poziotinib, and dacomitinib). In some embodiments, the EGFR inhibitor is a third generation EGFR TKI (e.g. osimertinib, WZ-4002, nazartinib, TAS-6417, and rociletinib). In some embodiments, the EGFR inhibitor is a fourth generation EGFR TKI (e.g. EAI405). In some embodiments, the EGFR inhibitor is a pan-mutation-selective EGFR TKI (e.g. TAS-6417). In some embodiments, the EGFR inhibitor is an irreversible pan-HER inhibitor that also inhibits EGFR (e.g. poziotinib). In some embodiments, the EGFR inhibitor is effective in inhibiting EGFR with exon 19 deletion or exon 21 L858R substitution mutations (e.g., dacomitinib, erlotinib, gefitinib, and osimertinib). In some embodiments, the EGFR inhibitor is effective in inhibiting EGFR with EGFR T790M (e.g., osimertinib, rociletinib, nazartinib, WZ-4002, and TAS-6471). In some embodiments, the EGFR inhibitor is effective in inhibiting EGFR with EGFR C797S or C797G mutations (e.g., EAI045). In some embodiments, the EGFR inhibitor is effective in inhibiting wild-type EGFR (e.g., afatinib, poziotinib, and dacomitinib). In some embodiments, the EGFR inhibitor binds to EGFR reversibly (e.g., erlotinib and gefitinib). In some embodiments, the EGFR inhibitor binds to EGFR irreversibly (e.g., afatinib, poziotinib, dacomitinib, osimertinib, rociletinib, nazartinib, WZ-4002, and TAS-6417).

In some embodiments, EGFR inhibitors may include, but are not limited to, erlotinib (Tarceva), gefitinib (Iressa), afatinib (Gilotrif), rociletinib (CO-1686), osimertinib (Tagrisso), olmutinib (Olita), naquotinib (ASP8273), nazartinib (EGF816), PF-06747775, icotinib (BPI-2009), neratinib (HKI-272; PB272); avitinib (ACOOIO), EAI045, tarloxotinib (TH-4000; PR-610), PF-06459988, tesevatinib (XL647; EXEL-7647; KD-019), transtinib, WZ-3146, WZ8040, CNX-2006, lapatinib (Tykerb), brigatinib (Alunbrig), sapitinib, CUDC-101, PD153035, pelitinib, AEE788 (NVP-AEE788), AST-1306, AZ5104, lifirafenib (BGB-283), canertinib, CL-387785 (EKI-785), norcantharadin, vandetanib (Caprelsa), dacomitinib (Vizimpro), WZ-4002, poziotinib, rociletinib, and TAS-6417 (CLN-081). In some embodiments, the EGFR-TKI is afatinib, dacomitinib, erlotinib, gefitinib, nazartinib, osimertinib, poziotinib, rociletinib, TAS-6417, or WZ-4002. In some embodiments, the EGFR inhibitor is selected from the group consisting of rac-(E)-N-[4-(3-chloro-4-fluoro-anilino)-7-[rac-(3S)-tetrahydrofuran-3-yl]oxy-quinazolin-6-yl]-4-(dimethylamino)but-2-enamide (afatinib), (E)-N-[4-(3-chloro-4-fluoro-anilino)-7-methoxy-quinazolin-6-yl]-4-(1-piperidyl)but-2-enamide (dacomitinib), N-(3-ethynylphenyl)-6,7-bis(2-methoxyethoxy)quinazolin-4-amine (erlotinib), N-(3-chloro-4-fluoro-phenyl)-7-methoxy-6-(3-morpholinopropoxy)quinazolin-4-amine (gefitinib), N-[7-chloro-1-[rac-(3R)-1-[rac-(E)-4-(dimethylamino)but-2-enoyl]azepan-3-yl]benzimidazol-2-yl]-2-methyl-pyridine-4-carboxamide (nazartinib), N-[2-[2-(dimethylamino)ethyl-methyl-amino]-4-methoxy-5-[[4-(1-methylindol-3-yl)pyrimidin-2-yl]amino]phenyl]prop-2-enamide (osimertinib), 1-[4-[4-(3,4-dichloro-2-fluoro-anilino)-7-methoxy-quinazolin-6-yl]oxy-1-piperidyl]prop-2-en-1-one (poziotinib), N-[3-[[2-[4-(4-acetylpiperazin-1-yl)-2-methoxy-anilino]-5-(trifluoromethyl)pyrimidin-4-yl]amino]phenyl]prop-2-enamide (rociletinib), N-[rac-(8S)-4-amino-6-methyl-5-(3-quinolyl)-8,9-dihydropyrimido[5,4-b]indolizin-8-yl]prop-2-enamide (TAS-6417), and N-[3-[5-chloro-2-[2-methoxy-4-(4-methylpiperazin-1-yl)anilino]pyrimidin-4-yl]oxyphenyl]prop-2-enamide (WZ-4002), and analogs or pharmaceutically acceptable salts thereof.

In some embodiments, the EGFR inhibitor in the pharmaceutical composition, method of treatment, or kits is afatinib. Afatinib is an irreversible, second generation EGFR TKI; see, e.g., Li et al. (2008) Oncegene 27(34):4702-11; Wang et al. (2014) Onocotarget 5(23):11971-85; Wong et al (2015) Am J Cancer Res. 5(12):3588-00. The free base of afatinib is chemically described as rac-(E)-N-[4-(3-chloro-4-fluoro-anilino)-7-[rac-(3S)-tetrahydrofuran-3-yl]oxy-quinazolin-6-yl]-4-(dimethylamino)but-2-enamide, having the structure below.

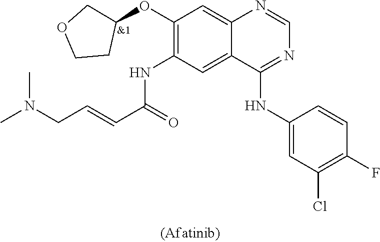 [SMILES: CN(C)C/C=C/C(=O)Nc1cc2c(Nc3ccc(F)c(Cl)c3)ncnc2cc1O[C@H]1CCOC1]

Description of afatinib and methods of making afatinib can be found in U.S. Pat. No. RE43431, the entirety of which is incorporated herein by reference.

Afatinib may be found in the form of the dimaleate salt. Afatinib dimaleate, also known as GILOTRIF®, is indicated for first-line treatment of patients with metastatic non-small cell lung cancer (NSCLC) whose tumors have non-resistant EGFR mutations as detected by an FDA-approved test and for treatment of patients with metastatic, squamous NSCLC progressing after platinum-based chemotherapy. Afatinib is available in 20 mg, 30 mg, and 40 mg (expressed as free base) tablets for oral use.

In some embodiments, the EGFR inhibitor in the pharmaceutical composition, method of treatment, or kits is dacomitinib. Dacomitinib is an irreversible, second generation EGFR TKI; see, e.g., Engelman et al. (2007) Cancer Res. 67(24):11924-32; Kalou et al. (2012) Mol Cancer Ther. 11(9):1978-87, the entirety of which is incorporated herein by reference. Dacomitinib is chemically described as (E)-N-[4-(3-chloro-4-fluoro-anilino)-7-methoxy-quinazolin-6-yl]-4-(1-piperidyl)but-2-enamide, having the structure below.

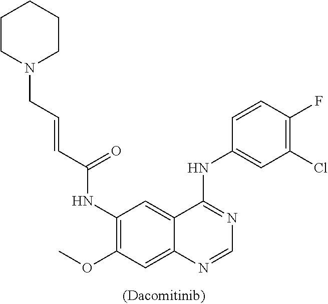 [SMILES: COc1cc2ncnc(Nc3ccc(F)c(Cl)c3)c2cc1NC(=O)/C=C/CN1CCCCC1]

Dacomitinib monohydrate, also known as VIZIMPRO®, is indicated for the treatment of metastatic NSCLC with EGFR exon 19 deletion or exon 21 L858R substitution mutations as detected by an FDA-approved test. Dacomitinib is available in 15 mg, 30 mg, and 45 mg tablets for oral use.

In some embodiments, the EGFR inhibitor in the pharmaceutical composition, method of treatment, or kits is erlotinib. Erlotinib is a first generation reversible EGFR TKI; see, e.g., Moyer et al (1997) Cancer Res. 57(21):4838-48; Ali et al. (2008) Mol Cancer Ther. 7(6):1708-19. The free base of erlotinib is chemically described as N-(3-ethynylphenyl)-6,7-bis(2-methoxyethoxy)quinazolin-4-amine, having the structure below.

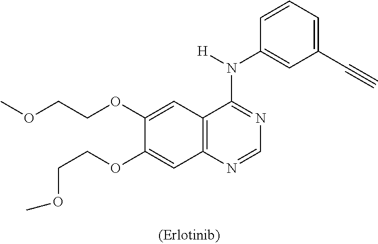 [SMILES: C#Cc1cccc(Nc2ncnc3cc(OCCOC)c(OCCOC)cc23)c1]

Description of erlotinib and methods of making erlotinib can be found in U.S. Pat. No. 5,747,498, the entirety of which is incorporated herein by reference.

Erlotinib may be found in the form of the hydrochloride salt of N-(3-ethynylphenyl)-6,7-bis(2-methoxyethoxy)quinazolin-4-amine. Erlotinib hydrochloride, also known as TARCEVA®, is indicated for the treatment of patients with locally advanced or metastatic NSCLC whose disease has not progressed after four cycles of platinum-based first-line chemotherapy; treatment of locally advanced or metastatic NSCLC after failure of at least one prior chemotherapy regimen; and first-line treatment of patients with locally advanced, unresectable or metastatic pancreatic cancer, in combination with gemcitabine. Erlotinib is available as oral 25 mg, 100 mg, and 150 mg tablets.

In some embodiments, the method of treating cancer in an individual in need thereof comprises administering to the individual a therapeutically effective amount of a combination of (i) a first therapy comprising an EGFR inhibitor (e.g., erlotinib), (ii) a second therapy comprising a PERK activator (e.g., IPA, MK-28, DHBDC, and CCT-020312), and (iii) a third therapy comprising an anti-cancer agent, such as a chemotherapy agent (e.g., gemcitabine, cisplatin, paclitaxel, etoposide, and pemetrexed).

In some embodiments, the EGFR inhibitor in the pharmaceutical composition, method of treatment, or kits is gefitinib. Gefitinib is a first generation reversible EGFR TKI; see, e.g., Wakeling et al. (2002) Cancer Res. 62(20):5749-54; Pedersen et al (2005) Br J Cancer 93(8):915-23; Moasser et al (2001) Cancer Res. 61(19):7184-8, the entirety of each is incorporated herein by reference. Gefitinib is chemically described as N-(3-chloro-4-fluoro-phenyl)-7-methoxy-6-(3-morpholinopropoxy)quinazolin-4-amine, having the structure below.

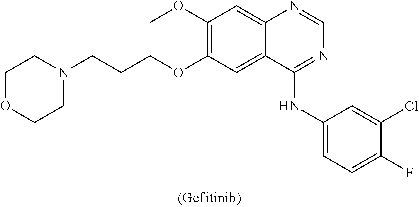 [SMILES: COc1cc2ncnc(Nc3ccc(F)c(Cl)c3)c2cc1OCCCN1CCOCC1]

Gefitinib, also known as IRESSA®, is FDA approved as a first-line treatment of patients with metastatic NSCLC whose tumors have EGFR exon 19 deletions or exon 21 L858R substitution mutations. Gefitinib is available in 250 mg (free base) tablets for oral once daily administration.

In some embodiments, the EGFR inhibitor in the pharmaceutical composition, method of treatment, or kits is nazartinib. Nazartinib, also known as EGF816 or NVS-816, is a third generation, irreversible, mutant-selective EGFR inhibitor targeting L858R, ex19del and T790M EGFR mutants; see, e.g., Lelais et al. (2016) J Med Chem. 59(14):6671-89; Jia et al (2016) Cancer Res. 76(6): 1591-602. Nazartinib is chemically described as N-[7-chloro-1-[rac-(3R)-1-[rac-(E)-4-(dimethylamino)but-2-enoyl]azepan-3-yl]benzimidazol-2-yl]-2-methyl-pyridine-4-carboxamide, having the structure below.

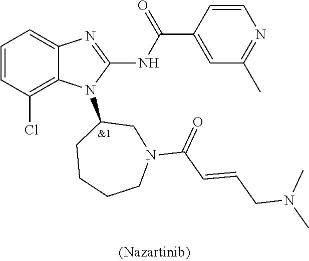 [SMILES: Cc1cc(C(=O)Nc2nc3cccc(Cl)c3n2[C@@H]2CCCCN(C(=O)/C=C/CN(C)C)C2)ccn1]

Description of nazartinib and methods of making nazartinib can be found in international patent application WO2013/184757, the entirety of which is incorporated herein by reference. Nazartinib is currently being evaluated in combination with TNO155 in patients with advanced EGFR mutant NSCLC (see, e.g., NCT03114319).

TNO155 is a highly potent, selective, orally efficacious, and first-in-class Sre homology-2 domain-containing protein tyrosine phosphatase-2 (SHP2) inhibitor currently in clinical trials for cancer. TNO155 is chemically described as (3S,4S)-8-(6-amino-5-((2-amino-3-chloropyridin-4-yl)thio)pyrazin-2-yl)-3-methyl-2-oxa-8-azaspiro[4.5]decan-4-amine. In some embodiments, the method of treating cancer in an individual in need thereof comprises administering to the individual a therapeutically effective amount of a combination of (i) a first therapy comprising an EGFR inhibitor (e.g., nazartinib), (ii) a second therapy comprising a PERK activator (e.g., IPA, MK-28, DHBDC, and CCT-020312), and (iii) a third therapy comprising a SHP2 inhibitor (e.g., TNO155).

In some embodiments, the EGFR inhibitor in the pharmaceutical composition, method of treatment, or kits is osimertinib. Osimertinib, also known as AZD9291, is a third generation EGFR TKI that binds irreversibly to EGFR proteins expressed by T790M mutation; it also binds irreversibly to EGFR with a L858R mutation and with an exon19 deletion; see, e.g. Cross et al. (2013) Mol Cancer Ther. 12, A109. The free base of osimertinib is chemically described as N-[2-[2-(dimethylamino)ethyl-methyl-amino]-4-methoxy-5-[[4-(1-methylindol-3-yl)pyrimidin-2-yl]amino]phenyl]prop-2-enamide, having the structure below.

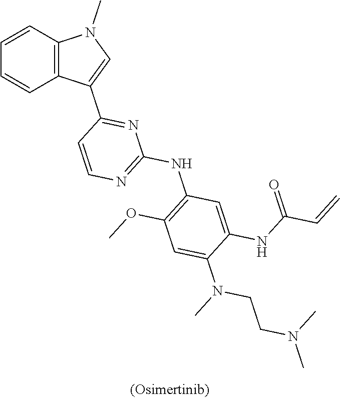 [SMILES: C=CC(=O)Nc1cc(Nc2nccc(-c3cn(C)c4ccccc34)n2)c(OC)cc1N(C)CCN(C)C]

Description of osimertinib and methods of making osimertinib can be found in international patent application WO2013/014448, the entirety of which is incorporated herein by reference.

Osimertinib may be found in the form of the mesylate salt N-[2-[2-(dimethylamino)ethyl-methyl-amino]-4-methoxy-5-[[4-(1-methylindol-3-yl)pyrimidin-2-yl]amino]phenyl]prop-2-enamide mesylate salt. Osimertinib mesylate is also known as TAGRISSO®. Osimertinib mesylate is indicated for the first-line treatment of adult patients with metastatic NSCLC whose tumors have EGFR exon 19 deletions or exon 21 L858R mutations; for the treatment of adult patients with metastatic EGFR T790M mutation positive NSCLC, whose disease has progressed on or after EGFR TKI therapy; or as adjuvant therapy after tumor resection in adult patients with NSCLC whose tumors have EGFR exon19 deletions or exon21 L858R mutations. Osimertinib is available in 40 mg and 80 mg (expressed as free base) tablets for oral once daily administration.

In some embodiments, the EGFR inhibitor in the pharmaceutical composition, method of treatment, or kits is poziotinib. Poziotinib is a second generation, pan-EGFR/HER inhibitor; see, e.g., Nam et al (2011) Cancer Lett. 28:302(2):155-65; Cha et al. (2012) Int J Cancer 130(10), 2445-2454. Poziotinib is chemically described as 1-[4-[4-(3,4-dichloro-2-fluoro-anilino)-7-methoxy-quinazolin-6-yl]oxy-1-piperidyl]prop-2-en-1-one, having the structure below.

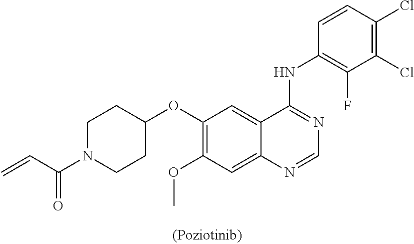 [SMILES: C=CC(=O)N1CCC(Oc2cc3c(Nc4ccc(Cl)c(Cl)c4F)ncnc3cc2OC)CC1]

Description of poziotinib and methods of making poziotinib can be found in U.S. Pat. No. 8,188,102 titled “Amide Derivative for Inhibiting the Growth of Cancer Cells”, the entirety of which is incorporated herein by reference. Poziotinib is being evaluated in patients with breast cancer and/or NSCLC in clinical trials such as NCT03066206, NCT03744715, and NCT02659514.

In some embodiments, the EGFR inhibitor in the pharmaceutical composition, method of treatment, or kits is rociletinib. Rociletinib is a third generation irreversible EGFR TKI that specifically targets the mutant forms of EGFR such as T790M; see, e.g., Walter et al. (2013) Cancer discov. 3(12):1404-15. Rociletinib is chemically described as N-[3-[[2-[4-(4-acetylpiperazin-1-yl)-2-methoxy-anilino]-5-(trifluoromethyl)pyrimidin-4-yl]amino]phenyl]prop-2-enamide, having the structure below.

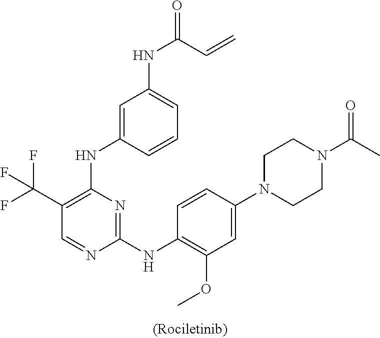 [SMILES: C=CC(=O)Nc1cccc(Nc2nc(Nc3ccc(N4CCN(C(C)=O)CC4)cc3OC)ncc2C(F)(F)F)c1]

Description of rociletinib and methods of making rociletinib can be found in U.S. patent application US2012/0149687 titled “Heterocyclic Compounds and Uses Thereof”, the entirety of which is incorporated herein by reference. Rociletinib has been evaluated in patients with NSCLC in clinical trials such as NCT02147990, NCT02322281, NCT0218630, and NCT01526928.

In some embodiments, the EGFR inhibitor in the pharmaceutical composition, method of treatment, or kits is TAS-6417. TAS-6417, also known as CLN-081, is a third generation, orally available, broad spectrum EGFR mutant inhibitor with activity against Ex20ins, exon19del, L858R, T790M, G719X, L861Q, and S768I mutations; see, e.g., Hasko et al. (2018) Mol Cancer Ther 17(8): 1648-1658; Udagawa et al. (2019) Mol Cancer Res. 17(11):2233-2243. TAS-6417 is chemically described as N-[rac-(8S)-4-amino-6-methyl-5-(3-quinolyl)-8,9-dihydropyrimido[5,4-b]indolizin-8-yl]prop-2-enamide, having the structure below.

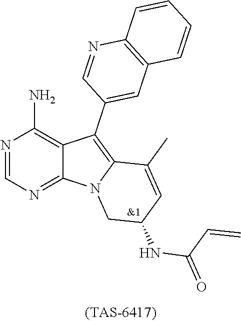 [SMILES: C=CC(=O)N[C@H]1C=C(C)c2c(-c3cnc4ccccc4c3)c3c(N)ncnc3n2C1]

Description of TAS-6417 and methods of making TAS-6417 can be found in international patent application WO2015/025936 titled “Novel Quinoline-Substituted Compound”, the entirety of which is incorporated herein by reference. TAS-6417 is being evaluated in patients with NSCLC in a phase 1/2a clinical trial (see NCT04036682).

In some embodiments, the EGFR inhibitor in the pharmaceutical composition, method of treatment, or kits is WZ-4002. WZ-4002 is a third generation, mutant-selective EGFR inhibitor for EGFR L858R/T790M; see, e.g., Zhou et al. (2009) Nature 462(7276), 1070-1074; Sakuma et al. (2012) Lab Invest. 92(3), 371-383; and Zannetti et al. (2012) J Nucl Med. 53(3), 443-450. WZ-4002 is chemically described as N-[3-[5-chloro-2-[2-methoxy-4-(4-methylpiperazin-1-yl)anilino]pyrimidin-4-yl]oxyphenyl]prop-2-enamide, having the structure below.

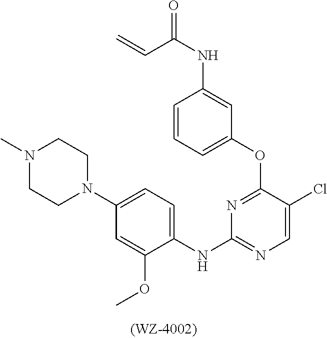 [SMILES: C=CC(=O)Nc1cccc(Oc2nc(Nc3ccc(N4CCN(C)CC4)cc3OC)ncc2Cl)c1]

Description of WZ-4002 and methods of making WZ-4002 can be found in international patent application WO2010/129053 titled “EGFR Inhibitors and Methods of Treating Disorders”, the entirety of which is incorporated herein by reference.

In some embodiments, the first therapy comprises an EGFR inhibitor selected from the group consisting of neratinib (HKI-272 or PB272), icotinib (BPI-2009), Olmutinib (Olita), Naquotinib (ASP8273), PF-06747775, avitinib, tarloxotinib, PF-06459988, tesevatinib (SL647, EXEL-7647, and KD-019), transtinib, WZ-3146, WZ8040, CNX-2006, EI045, brigatinib, vandetanib (Caprelsa), norcantharadin, CL-387785 (EKI-785), Canertinib, lifirafenib (BGB-283), AZ5104, AST-1306, AEE788, pelitinib, PD153035, CUDC-101, sapitinib (AZD8931), and lapatinib (Tykerb).

### PERK Activator

In some embodiments, the second therapy comprises any agent that demonstrates EIF-2 kinase (e.g., eIF2 α kinases) activation activity such as an ISR activator (e.g., a PERK, GCN2, PKR, and/or HRI activator). In some embodiments, the agent in the second therapy increases phosphorylation of elF2 α. In some embodiments, the agent in the second therapy promotes expression of the ATF4 gene. In some embodiments, the agent in the second therapy is a PERK activator (e.g., IPA, MK-28, CCT-020312, and DHBDC). In some embodiments, the PERK activator selectively activates PERK over GCN2, PKR, or HRI. In some embodiments, the PERK activator activates PERK, GCN2, PKR, HRI, or any combination thereof. In some embodiments, the second therapy comprises a PERK activator selected from the group consisting of 1-(4-((4-((5-cyclopropyl-1H-pyrazol-3-yl)amino)pyrimidin-2-yl)amino)phenyl)-3-(4-(methylthio)phenyl)urea (IPA), 4-[(E)-[(4,6-diphenylpyrimidin-2-yl)-methyl-hydrazono]methyl]benzene-1,2-diol (MK-28), 3-(2,3-dihydrobenzo[b][1,4]dioxin-6-yl)-5,7-dihydroxy-4H-chromen-4-one (DHBDC), 6-bromo-3-[3-(4-bromophenyl)-2-[3-(diethylamino)propanoyl]-3,4-dihydropyrazol-5-yl]-4-phenyl-1H-quinolin-2-one (CCT-020312), and pharmaceutically acceptable salts thereof.

In some embodiments, the PERK activator is CCT020312. CCT020312 is a selective EIF2AK3/PERK activator; see, e.g., Stockwell et al. (2012) PloS One. 7(1):e28568 and international patent application WO 2016/024010 the entirety of which is incorporated herein by reference. CCT020312 is chemically described as 6-bromo-3-[3-(4-bromophenyl)-2-[3-(diethylamino)propanoyl]-3,4-dihydropyrazol-5-yl]-4-phenyl-1H-quinolin-2-one, having the chemical structure below

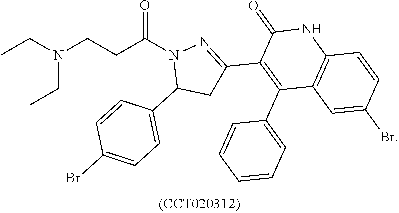 [SMILES: CCN(CC)CCC(=O)N1N=C(c2c(-c3ccccc3)c3cc(Br)ccc3[nH]c2=O)CC1c1ccc(Br)cc1]

CCT020312 is commercially available as a yellowish-white solid (MilliporeSigma, CAS No. 324759-76-4).

In some embodiments, the PERK activator in the pharmaceutical composition, method of treatment, or kits is MK-28. MK-28 is a selective PERK activator, see, e.g., Ganz et al. (2020) Sci Rep. 10(1):6875. MK-28 is chemically described as 4-[(E)-[(4,6-diphenylpyrimidin-2-yl)-methyl-hydrazono]methyl]benzene-1,2-diol, having the structure below.

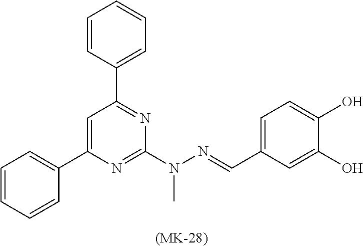

MK-28 is disclosed in international patent application WO 2017/216792, the entirety of which is incorporated herein by reference. Description of MK-28 and methods of making MK-28 can be found, e.g., on page 46 synthesis of compound 3 MK-28, of the above mentioned reference. MK-28 is also commercially available as a powder (MedChemExpress, CAS No. 864388-65-8).

In some embodiments, the PERK activator in the pharmaceutical composition, method of treatment, or kits is DHBDC. DHBDC is a dual activator of PKR and PERK, see e.g., Bai, H., et al. (2013) ChemBioChem 14, 1255 titled “Dual Activators of Protein Kinase R (PKR) and Protein Kinase R-Like Kinase (PERK) Identify Common and Divergent Catalytic Target”. DHBDC is described chemically as 3-(2,3-dihydrobenzo[b][1,4]dioxin-6-yl)-5,7-dihydroxy-4H-chromen-4-one having the structure formula:

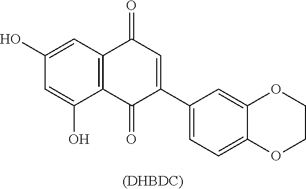

DHBDC is commercially available as a pink powder (MilliporeSigna, CAS No. 101068-35-3).

In some embodiments, the PERK activator in the pharmaceutical composition, method of treatment, or kits is IPA. IPA is a PERK activator, chemically described as 1-(4-((4-((5-cyclopropyl-1H-pyrazol-3-yl)amino)pyrimidin-2-yl)amino)phenyl)-3-(4-(methylthio)phenyl)urea, having the structure below.

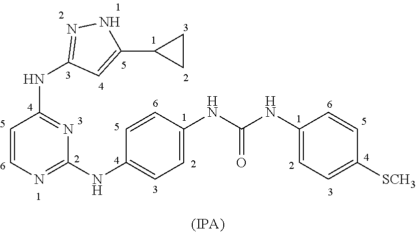 [SMILES: CSc1ccc(NC(=O)Nc2ccc(Nc3nccc(Nc4cc(C5CC5)[nH]n4)n3)cc2)cc1]

IPA is disclosed in Mendez et al. (2015) eLife 4:e05434 titled “Endoplasmic Reticulum Stress-Independent Activation of Unfolded Protein Response Kinases by a Small Molecule ATP-Mimic”, the entirety of which is incorporated herein by reference. Description of IPA and methods of making IPA can be found, e.g., on page 21 synthesis of cmp6 (IPA), of the above-mentioned reference.

### Methods

Methods of treating cancer in an individual in need thereof with a combination of an EGFR inhibitor and a PERK activator are provided herein. In some embodiments, treatment of cancer in an individual in need thereof comprises administering to the individual a therapeutically effective amount of a combination of (i) a first therapy comprising an EGFR inhibitor, and (ii) a second therapy comprising a PERK activator. In some embodiments, the first and second therapies have a synergistic effect. In some embodiments, the first and second therapies have at least an additive effect. In some embodiments, combination of a PERK activator with an EGFR inhibitor results in increased EGFR inhibitor efficacy. In some embodiments combination of a PERK activator with an EGFR inhibitor results in increased expression of an ER protein processing gene. As is understood in the clinical context, a therapeutically effective dosage of a drug, compound or pharmaceutical composition that contains an EGFR inhibitor may be achieved in conjunction with another drug, compound or pharmaceutical composition that contains one or more compounds that activates PERK (e.g. CCT-020312, MK-28, IPA, or DHBDC). Thus, a therapeutically effective amount may be considered in the context of administering one or more therapeutic agents, and a single agent may be considered to be given in a therapeutically effective amount if, in conjunction with one or more other agents, a desirable or beneficial result may be or is achieved. The compounds in a combination therapy of the invention may be administered sequentially, simultaneously, or continuously using the same or different routes of administration for each compound. Thus, a therapeutically effective amount of a combination therapy includes an amount of the first therapy and an amount of the second therapy that when administered sequentially, simultaneously, or continuously produces a desired outcome. Suitable doses of any of the co-administered compounds may optionally be lowered due to the combined action (e.g., additive or synergistic effects) of the compounds.

In some embodiments, the disclosed compounds, and any combinations thereof, are for use as therapeutically active substance.

In some embodiments, the disclosed compounds, and any combinations thereof, are for the therapeutic and/or prophylactic treatment of cancer.

In some embodiments, the disclosed compounds, and any combinations thereof, are for the preparation of a medicament for the therapeutic treatment of cancer.

In some embodiments, the disclosed compounds, and any combinations thereof, are for use in the therapeutic treatment of cancer.

In one aspect, the present disclosure is directed to the use of a combination comprising one or more EGFR inhibitors and one or more PERK activators in the manufacture of a medicament for use in the treatment of cancer in a subject in need thereof.

In one aspect, the present disclosure is directed to compositions comprising: (i) one or more EGFR inhibitors; and (ii) one or more PERK activators for use in the treatment of cancer.

In various embodiments, treatment with the combination of the first and second therapies may result in an additive or even synergistic (e.g., greater than additive) result compared to administration of either therapy alone. For example, if agent “A” causes 10-fold decrease in cell viability and agent “B” causes 10-fold decrease in cell viability, the (non-synergistic) sum of such effects would be 20-fold decrease in cell viability. If, in fact, the combination of agents A and B results in greater than 20 fold decrease in cell viability, their combined effect would be considered synergistic.

In some embodiments, a lower amount of each pharmaceutically active compound is used as part of a combination therapy compared to the amount generally used for individual therapy. Preferably, the same or greater therapeutic benefit is achieved using a combination therapy than by using any of the individual compounds alone. In some embodiments, the same or greater therapeutic benefit is achieved using a smaller amount (e.g., a lower dose or a less frequent dosing schedule) of a pharmaceutically active compound in a combination therapy than the amount generally used for individual therapy. Preferably, the use of a small amount of pharmaceutically active compound results in a reduction in the number, severity, frequency, or duration of one or more side effects associated with the compound. In some embodiments, the amount of the second therapy or the combined therapy is an amount sufficient to increase efficacy of the EGFR inhibitor. In some embodiments, the amount of the first therapy, the second therapy, or the combined therapy is an amount sufficient to increase expression of an ER protein processing genes in the individual.

To achieve treatment, the amount (e.g. therapeutically effective amount) of the EGFR inhibitor may be lowered (e.g. lower doses and/or less frequent administration) when administered with the PERK activator than when the EGFR inhibitor is administered without the PERK activator. In some embodiments, the therapeutically effective amount of the EGFR inhibitor when administered with the PERK activator may be a sub-efficacious amount in a monotherapy (e.g., a therapy comprises the EGFR inhibitor only).

Efficacy of an EGFR inhibitor may be assessed by methods, including but not limited to, pre-clinical and/or clinical assessments, in vitro assays with purified enzyme, cell-based assays, animal models, or human testing. Clinical assessments may include progression-free survival (PFS) as assessed by an independent review committee (IRC) per RECIST v1.1, overall response rate (ORR), duration of response (DOR), and overall survival (OS). Cell-based assessments may include cell viability, cell proliferation, colony formation, cytotoxicity, cytostatsis, induction of apoptosis, and cell cycle arrest. Efficacy of an EGFR inhibitor may be assessed (e.g. by decrease in cell viability) after a treatment of EGFR inhibitor, a treatment of PERK activator, or a combination thereof.

In some embodiments, the first therapy comprises an EGFR inhibitor selected from the group consisting of afatinib, dacomitinib, erlotinib, gefitinib, nazartinib, osimertinib, poziotinib, rociletinib, TAS-6417, WZ-4002, and pharmaceutically acceptable salts thereof, and the second therapy comprises a PERK activator selected from the group consisting of IPA, MK-28 DHBDC, CCT-020312, and pharmaceutically acceptable salts thereof. In some embodiments, the first therapy comprises an EGFR inhibitor selected from the group consisting of afatinib, dacomitinib, erlotinib, gefitinib, nazartinib, osimertinib, poziotinib, rociletinib, TAS-6417, WZ-4002, and pharmaceutically acceptable salts thereof, and the second therapy comprises IPA or a pharmaceutically acceptable salt thereof. In some embodiments, the first therapy comprises an EGFR inhibitor selected from the group consisting of afatinib, dacomitinib, erlotinib, gefitinib, nazartinib, osimertinib, poziotinib, rociletinib, TAS-6417, WZ-4002, and pharmaceutically acceptable salts thereof, and the second therapy comprises MK-28 or a pharmaceutically acceptable salt thereof. In some embodiments, the first therapy comprises an EGFR inhibitor selected from the group consisting of afatinib, dacomitinib, erlotinib, gefitinib, nazartinib, osimertinib, poziotinib, rociletinib, TAS-6417, WZ-4002, and pharmaceutically acceptable salts thereof, and the second therapy comprises DHBDC or a pharmaceutically acceptable salt thereof. In some embodiments, the first therapy comprises an EGFR inhibitor selected from the group consisting of afatinib, dacomitinib, erlotinib, gefitinib, nazartinib, osimertinib, poziotinib, rociletinib, TAS-6417, WZ-4002, and pharmaceutically acceptable salts thereof, and the second therapy comprises CCT-020312 or a pharmaceutically acceptable salt thereof.

In some embodiments, the first therapy is a first generation EGFR TKI (e.g. erlotinib, gefitinib, and pharmaceutically acceptable salts thereof) and the second therapy comprises a PERK activator (e.g. IPA, MK-28 DHBDC, CCT-020312, and pharmaceutically acceptable salts thereof). In some embodiments, the first therapy is a second generation EGFR TKI (e.g., afatinib, poziotinib, dacomitinib, and pharmaceutically acceptable salts thereof) and the second therapy comprises a PERK activator (e.g. IPA, MK-28 DHBDC, CCT-020312, and pharmaceutically acceptable salts thereof). In some embodiments, the first therapy is a third generation EGFR TKI (e.g. osimertinib, rociletinib, nazartinib, WZ-4002, TAS-6417, and pharmaceutically acceptable salts thereof) and the second therapy comprises a PERK activator (e.g. IPA, MK-28 DHBDC, CCT-020312, and pharmaceutically acceptable salts thereof).

In embodiments, the amount of the first therapy and the amount of the second therapy administered to a subject are present in a molar ratio of 1:1 to 1:10, 1:2 to 1:20, 1:10 to 1:100, 1:20 to 1:200, 1:50 to 1:500, 1:200 to 1:2000, or 1:500 to 1:5000. In some embodiments, the amount of the first therapy and the amount of the second therapy in a kit are present in a molar ratio of 10:1 to 1:100, 5:1 to 1:200, 1:1 to 1:1000, 1:2 to 1:2000, 1:5 to 1:5000, 1:20 to 1:20000, or 1:50 to 1:50000.

In some embodiments, the first and second therapies are administered simultaneously, with a time separation of no more than 5 minutes (such as no more than about any of 1, 2, 3, or 4 minutes). When the compounds are administered simultaneously, the first and second therapies may be contained in the same composition (e.g., a composition comprising both an EGFR inhibitor and a PERK activator) or in separate compositions (e.g., an EGFR inhibitor is contained in one composition and a PERK activator is contained in another composition). In some embodiments, the first and second therapies are administered sequentially, with a time separation of more than about 5 minutes (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks). In some embodiments, the EGFR inhibitor is administered before the PERK activator. In some embodiments, the PERK activator is administered before the EGFR inhibitor. The first and second therapies are contained in separate compositions, which may be contained in the same or different packages or kits.

In some embodiments, treatment may comprise one or more additional therapy (e.g., a third anti-cancer therapy), such as chemotherapy, radiation therapy, surgery, targeted therapy, immunotherapy, hormone therapy, and stem cell or bone marrow transplant. In some embodiments, the one or more additional therapy comprises one or more anti-cancer agent, such as a SHP2 inhibitor, MEK inhibitor, VEGFR inhibitor, anti-VEGFR2 antibody, KDR antibody. AKT inhibitors, PDK-I inhibitors, PI3K inhibitors, c-kit/Kdr tyrosine kinase inhibitor, Ber-Abl tyrosine kinase inhibitor. VEGFR2 inhibitor, PDGFR-beta inhibitor, KIT inhibitor. Flt3 tyrosine kinase inhibitor. PDGF receptor family inhibitor, Flt3 tyrosine kinase inhibitor, RET tyrosine kinase receptor inhibitor. VEGF-3 receptor antagonist, Raf protein kinase inhibitor, angiogenesis inhibitor, Erb2 inhibitor, mTOR inhibitor, IGF-IR antibody. NFKB inhibitor, proteasome inhibitor, chemotherapy agent, or glucose reduction agent. In some embodiments, the third therapy comprises one or more chemotherapy agents (e.g., gemcitabine). In some embodiments, the third therapy comprises one or more SHP2 inhibitors (e.g., TNO155).

### Cancers

In various embodiments of this invention, cancer includes but is not limited to, carcinoma, lymphoma, blastoma, sarcoma, and leukemia. More particular examples of such cancers include but are not limited to squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Examples of cancer may include primary tumors of any of the above types of cancer or metastatic tumors at a second site derived from any of the above types of cancer.

In some embodiments, the cancer is associated with over-expression of EGFR kinase. Cancers associated with over-expression of EGFR kinase include, but not limited to, breast cancer, lung cancer, colorectal cancer, ovarian cancer, renal cell cancer, bladder cancer, head and neck cancers, glioblastomas, and astrocytomas. A cancer associated with over-expression of EGFR kinase may or may not be an EGFR-mutant cancer. In some embodiments, the cancer associated with over-expression of EGFR kinase is an EGFR-mutant cancer. In some embodiments, the cancer associated with over-expression of EGFR kinase is not an EGFR-mutant cancer.

When an individual is diagnosed with a cancer (e.g. EGFR-mutant cancer), the individual may be further tested to determine the presence of one or more EGFR mutations (e.g. ex19del, L858R, L861Q, T790M, C797S, C797G, T854A, D761Y, L747S, G719X, L861X, V8343I, V769M, ex20ins, and A871E). For example, an individual diagnosed with NSCLC may require further testing to determine whether the NSCLC is associated with any EGFR mutations, such as EGFR exon 19 deletions, exon 21 L858R mutations, and/or T790M mutation. In some embodiments, the individual in need of treatment is diagnosed with NSCLC, prostate cancer, breast cancer, pancreatic cancer, or colon cancer. In some embodiments, the individual is diagnosed with NSCLC, wherein the NSCLC is metastatic NSCLC or locally advanced NSCLC. In some embodiments, the individual is diagnosed with EGFR exon 19 deletions positive NSCLC or exon 21 L858R mutations positive NSCLC. In some embodiments, the individual is diagnosed with EGFR T790M mutation positive NSCLC. In some embodiments, the individual is a human. In some embodiments, the individual is an adult. In some embodiments, the individual is a senior adult. In some embodiments, the individual is a juvenile.

### Composition

One or more compounds described herein such as the first therapy and/or the second therapy can be used in the preparation of a composition, such as a pharmaceutical composition, by combining the compound or compounds as an active ingredient with a pharmacologically acceptable carrier, which are known in the art. Depending on the therapeutic form of the system (e.g., transdermal patch vs. oral tablet), the carrier may be in various forms. In addition, pharmaceutical preparations may contain one or more excipients such as preservatives, solubilizers, stabilizers, re-wetting agents, emulgators, sweeteners, dyes, adjusters, salts for, e.g., the adjustment of osmotic pressure, buffers, coating agents and antioxidants. Suitable excipients may be found, e.g., in Remington's Pharmaceutical Sciences, Mack Publishing Company, Philadelphia, PA, 20^{th} ed. (2000), which is incorporated herein by reference. Preparations comprising a combination therapy may also contain other substances that have valuable therapeutic properties. Pharmaceutically active compounds in a combination therapy can be prepared as part of the same or different compositions to be administered together or separately. Therapeutic forms may be represented by a usual standard dose and may be prepared by a known pharmaceutical method. Suitable doses of any of the co-administered compounds may optionally be lowered due to the combined action (e.g., additive or synergistic effects) of the compounds.

In embodiments, the amount of the first therapy and the amount of the second therapy in a composition are present in a molar ratio of 1:1 to 1:10, 1:2 to 1:20, 1:10 to 1:100, 1:20 to 1:200, 1:50 to 1:500, 1:200 to 1:2000, or 1:500 to 1:5000. In some embodiments, the amount of the first therapy and the amount of the second therapy in a composition are present in a molar ratio of 10:1 to 1:100, 5:1 to 1:200, 1:1 to 1:1000, 1:2 to 1:2000, 1:5 to 1:5000, 1:20 to 1:20000, or 1:50 to 1:50000.

In some embodiments, the amount of the first therapy, the second therapy, or the combined therapy in a pharmaceutical composition is an amount sufficient to increase the EGFR inhibitor efficacy. In some embodiments, the EGFR inhibitor efficacy increases by at least 10%, 20%, 30%, 40%, 50%, 60%, 70%, 80%, 90%, 100%, 500%, 800%, 1000%, 1500%, 2000%, and 3000% as compared to the corresponding efficacy in the same subject prior to treatment or compared to the corresponding efficacy in other subjects not receiving the combination therapy. In some embodiments, the amount of the first therapy, the second therapy, or the combined therapy in a pharmaceutical composition is an amount sufficient to produce a desired therapeutic outcome (e.g., reducing the severity or duration of, stabilizing the severity of, or eliminating one or more symptoms of cancer). In various embodiments, the amount of the first therapy, the second therapy, or the combined therapy in a pharmaceutical composition is an amount sufficient to treat cancer when administered to an individual who is diagnosed with cancer (e.g., NSCLC, prostate cancer, breast cancer, colon cancer, and pancreatic cancer).

The first and second compounds of a combination therapy may be combined with a pharmaceutically acceptable carrier to produce a pharmaceutical composition. In some embodiments, the pharmaceutical composition comprises a first therapy comprising an EGFR inhibitor, a second therapy comprising a PERK activator (e.g., IPA, MK-28, DHBDC, CCT-020312, and pharmaceutically acceptable salts thereof), and a pharmaceutically acceptable carrier. In some embodiments, the first therapy of the pharmaceutical composition comprises an EGFR inhibitor selected from the group consisting of afatinib, dacomitinib, erlotinib, gefitinib, nazartinib, osimertinib, poziotinib, rociletinib, TAS-6417, WZ-4002, and pharmaceutically acceptable salts thereof, and the second therapy comprises a PERK activator selected from the group consisting of IPA, MK-28 DHBDC, CCT-020312, and pharmaceutically acceptable salts thereof. In some embodiments, the first therapy of the pharmaceutical composition comprises an EGFR inhibitor selected from the group consisting of afatinib, dacomitinib, erlotinib, gefitinib, nazartinib, osimertinib, poziotinib, rociletinib, TAS-6417, WZ-4002, and pharmaceutically acceptable salts thereof, and the second therapy comprises IPA or a pharmaceutically acceptable salt thereof. In some embodiments, the first therapy of the pharmaceutical composition comprises an EGFR inhibitor selected from the group consisting of afatinib, dacomitinib, erlotinib, gefitinib, nazartinib, osimertinib, poziotinib, rociletinib, TAS-6417, WZ-4002, and pharmaceutically acceptable salts thereof, and the second therapy comprises MK-28 or a pharmaceutically acceptable salt thereof. In some embodiments, the first therapy of the pharmaceutical composition comprises an EGFR inhibitor selected from the group consisting of afatinib, dacomitinib, erlotinib, gefitinib, nazartinib, osimertinib, poziotinib, rociletinib, TAS-6417, WZ-4002, and pharmaceutically acceptable salts thereof, and the second therapy comprises DHBDC or a pharmaceutically acceptable salt thereof. In some embodiments, the first therapy of the pharmaceutical composition comprises an EGFR inhibitor selected from the group consisting of afatinib, dacomitinib, erlotinib, gefitinib, nazartinib, osimertinib, poziotinib, rociletinib, TAS-6417, WZ-4002, and pharmaceutically acceptable salts thereof, and the second therapy comprises CCT-020312 or a pharmaceutically acceptable salt thereof.

In some embodiments, the first therapy of the pharmaceutical composition is a first generation EGFR TKI (e.g. erlotinib, gefitinib, and pharmaceutically acceptable salts thereof) and the second therapy comprises a PERK activator (e.g. IPA, MK-28 DHBDC, CCT-020312, and pharmaceutically acceptable salts thereof). In some embodiments, the first therapy of the pharmaceutical composition is a second generation EGFR TKI (e.g., afatinib, poziotinib, dacomitinib, and pharmaceutically acceptable salts thereof) and the second therapy comprises a PERK activator (e.g. IPA, MK-28 DHBDC, CCT-020312, and pharmaceutically acceptable salts thereof). In some embodiments, the first therapy of the pharmaceutical composition is a third generation EGFR TKI (e.g. osimertinib, rociletinib, nazartinib, WZ-4002, TAS-6417, and pharmaceutically acceptable salts thereof) and the second therapy comprises a PERK activator (e.g. IPA, MK-28 DHBDC, CCT-020312, and pharmaceutically acceptable salts thereof).

In some embodiments, the composition may comprise a unit dosage form of a first therapy comprising an EGFR inhibitor and a unit dosage form of a second therapy comprising a PERK activator (e.g., IPA, MK-28, DHBDC, CCT-020312, and pharmaceutically acceptable salts thereof). The compound in one or more unit dosage forms of the first therapy, the compound in one or more unit dosage forms of the second therapy, and/or the compounds in one or more unit dosage forms of a combination thereof, may be present in a therapeutically effective amount to treat cancer (e.g., NSCLC, prostate cancer, breast cancer, colon cancer, and pancreatic cancer). In embodiments, the compound of the first therapy is present in a single unit dosage form in a therapeutically effective amount to treat cancer (e.g., NSCLC, prostate cancer, breast cancer, colon cancer, and pancreatic cancer). In embodiments, the compound of the second therapy is present in a single unit dosage form in a therapeutically effective amount to treat cancer (e.g., NSCLC, prostate cancer, breast cancer, colon cancer, and pancreatic cancer).

In some aspects, the combinations and compositions of the present disclosure are for use in a combination therapy for the treatment of cancer (e.g., NSCLC, prostate cancer, breast cancer, colon cancer, and pancreatic cancer) in combination with one or more other therapeutic agents. In a further aspect, the combinations and compositions of the present disclosure are for use in a combination therapy for the treatment of NSCLC.

### Kits

The invention further provides kits comprising one or more compounds as described herein. The kits may employ any of the compounds disclosed herein and instructions for use. In some embodiments, the kit includes a first therapy comprising an EGFR inhibitor and a second therapy comprising a PERK activator. The kits may be used for any one or more of the uses described herein, and, accordingly, may contain instructions for any one or more of the stated uses (e.g., treating cancer).

In various embodiments, kit includes an EGFR inhibitor (e.g., a first generation EGFR TKI, a second generation EGFR TKI, a third generation EGFR TKI, a fourth generation EGFR TKI, a reversible EGFR TKI, or an irreversible EGFR TKI). The EGFR inhibitor is a small molecular EGFR TKI, wherein the EGFR TKI may be in any acceptable unit dosage form. In some embodiments, kit includes a PERK activator. The PERK activator is 1-(4-((4-((5-cyclopropyl-1H-pyrazol-3-yl)amino)pyrimidin-2-yl)amino)phenyl)-3-(4-(methylthio)phenyl)urea (IPA), 4-[(E)-[(4,6-diphenylpyrimidin-2-yl)-methyl-hydrazono]methyl]benzene-1,2-diol (MK-28), 3-(2,3-dihydrobenzo[b][1,4]dioxin-6-yl)-5,7-dihydroxy-4H-chromen-4-one (DHBDC), 6-bromo-3-[3-(4-bromophenyl)-2-[3-(diethylamino)propanoyl]-3,4-dihydropyrazol-5-yl]-4-phenyl-1H-quinolin-2-one (CCT-020312), or any pharmaceutically acceptable salts thereof.

In embodiments, the EGFR inhibitor of kit is selected from the group consisting of afatinib, dacomitinib, erlotinib, gefitinib, nazartinib, osimertinib, poziotinib, rociletinib, TAS-6417, WZ-4002, and pharmaceutically acceptable salts thereof, and the second therapy comprises a PERK activator selected from the group consisting of IPA, MK-28 DHBDC, CCT-020312, and pharmaceutically acceptable salts thereof. In some embodiments, the EGFR inhibitor of kit is selected from the group consisting of afatinib, dacomitinib, erlotinib, gefitinib, nazartinib, osimertinib, poziotinib, rociletinib, TAS-6417, WZ-4002, and pharmaceutically acceptable salts thereof, and the PERK activator comprises IPA or a pharmaceutically acceptable salt thereof. In some embodiments, the EGFR inhibitor of kit is selected from the group consisting of afatinib, dacomitinib, erlotinib, gefitinib, nazartinib, osimertinib, poziotinib, rociletinib, TAS-6417, WZ-4002, and pharmaceutically acceptable salts thereof, and the PERK activator comprises MK-28 or a pharmaceutically acceptable salt thereof. In some embodiments, the EGFR inhibitor of kit is selected from the group consisting of afatinib, dacomitinib, erlotinib, gefitinib, nazartinib, osimertinib, poziotinib, rociletinib, TAS-6417, WZ-4002, and pharmaceutically acceptable salts thereof, and the PERK activator comprises DHBDC or a pharmaceutically acceptable salt thereof. In some embodiments, the EGFR inhibitor of kit is selected from the group consisting of afatinib, dacomitinib, erlotinib, gefitinib, nazartinib, osimertinib, poziotinib, rociletinib, TAS-6417, WZ-4002, and pharmaceutically acceptable salts thereof, and the PERK activator comprises CCT-020312 or a pharmaceutically acceptable salt thereof.

In some embodiments, the kit comprises a first generation EGFR TKI (e.g. erlotinib, gefitinib, and pharmaceutically acceptable salts thereof) and a PERK activator (e.g. IPA, MK-28 DHBDC, CCT-020312, and pharmaceutically acceptable salts thereof). In some embodiments, the kit comprises a second generation EGFR TKI (e.g., afatinib, poziotinib, dacomitinib, and pharmaceutically acceptable salts thereof) and a PERK activator (e.g. IPA, MK-28 DHBDC, CCT-020312, and pharmaceutically acceptable salts thereof). In some embodiments, the kit comprises a third generation EGFR TKI (e.g. osimertinib, rociletinib, nazartinib, WZ-4002, TAS-6417, and pharmaceutically acceptable salts thereof) and a PERK activator (e.g. IPA, MK-28 DHBDC, CCT-020312, and pharmaceutically acceptable salts thereof).

In embodiments, the amount of the first therapy and the amount of the second therapy in a kit are present in a molar ratio of 1:1 to 1:10, 1:2 to 1:20, 1:10 to 1:100, 1:20 to 1:200, 1:50 to 1:500, 1:200 to 1:2000, or 1:500 to 1:5000. In some embodiments, the amount of the first therapy and the amount of the second therapy in a kit are present in a molar ratio of 10:1 to 1:100, 5:1 to 1:200, 1:1 to 1:1000, 1:2 to 1:2000, 1:5 to 1:5000, 1:20 to 1:20000, or 1:50 to 1:50000.

In some embodiments, the amount of the first therapy, the second therapy, or the combined therapy in a kit is an amount sufficient to increase the EGFR inhibitor efficacy. In some embodiments, the EGFR inhibitor efficacy increases by at least or about 10%, 20%, 30%, 40%, 50%, 60%, 70%, 80%, 90%, 100%, 500%, 800%, 1000%, 1500%, 2000%, and 3000% as compared to the corresponding efficacy in the same subject prior to treatment or compared to the corresponding efficacy in other subjects not receiving the combination therapy. In some embodiments, the amount of the first therapy, the second therapy, or the combined therapy in a kit is an amount sufficient to produce a desired therapeutic outcome (e.g., reducing the severity or duration of, stabilizing the severity of, or eliminating one or more symptoms of cancer). In various embodiments, the amount of the first therapy, the second therapy, or the combined therapy in a kit is an amount sufficient to prevent or reduce the severity of one or more future symptoms of cancer when administered to an individual who is susceptible and/or who may develop cancer (e.g., NSCLC, prostate cancer, breast cancer, colon cancer, and pancreatic cancer).

Kits generally comprise suitable packaging. The kits may comprise one or more containers comprising any compound described herein. Suitable packaging includes, but is not limited to, vials, bottles, jars, flexible packaging (e.g., plastic bags), and the like. Each component (if there is more than one component) can be packaged in separate containers or some components can be combined in one container where cross-reactivity and shelf life permit. Kits may optionally provide additional components such as buffers.

The kits may optionally include a set of instructions, generally written instructions, although electronic storage media (e.g., magnetic diskette or optical disk) containing instructions are also acceptable, relating to the use of component(s) of the methods of the present invention (e.g., treating cancer). The instructions included with the kit generally include information as to the components and their administration to an individual, such as information regarding dosage, dosing schedule, and route of administration.

The containers may be unit dosage forms, bulk packages (e.g., multi-dose packages) or sub-unit doses. For example, kits may be provided that contain sufficient dosages of an EGFR inhibitor and/or a PERK activator to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

### EXAMPLES

### Example 1. Synergistic Effect of EGFR Inhibitor in Combination with PERK Activator

PC9 cells were seeded in replicates in 96-well plates and allowed to attach overnight. The following day cells were treated with the indicated compounds or compound combinations at the indicated concentrations. Cell survival was measured 72 h post-treatment using CellTiter-Glo Luminescent Cell Viability Assay (Promega, G7572). Quantification of the fold decrease in cell viability was determined for single agent activity and for combinations relative to the DMSO-treated control (as shown in Table 1A and 1B). Mean (u) and standard deviation (o) was determined for biological triplicates using Microsoft Excel. Tested PERK activators concentrations are shown in Table 2. Tested EGFR inhibitors concentrations are shown in Table 3. As shown in Table 1A and 1B, synergy as measured in fold decrease in cell viability was observed across the tested combinations of EGFR inhibitors and PERK activators in at least one tested concentration of the EGFR inhibitors.

TABLE 1A

| | | | Low | High | | CCT-020312 + | CCT-020312 + | | DHBDC + | DHBDC + |
|---|---|---|---|---|---|---|---|---|---|---|
| | | DMSO | EGFRi | EGFRi | CCT-020312 | Low EGFRi | High EGFRi | DHBDC | Low EGFRi | High EGFRi |
| Afatinib | μ | 1.0 | 11.7 | 14.7 | 3.4 | 570.6 | 1165.6 | 2.5 | 64.4 | 66.6 |
| | σ | 0.07 | 0.8 | 0.9 | 0.2 | 117.3 | 104.0 | 0.3 | 5.4 | 2.1 |
| Dacomitinib | μ | 1.0 | 13.6 | 14.5 | 4.6 | 731.1 | 1325.3 | 2.5 | 66.5 | 85.2 |
| | σ | 0.05 | 1.0 | 1.2 | 0.5 | 144.8 | 263.1 | 0.1 | 5.5 | 3.9 |
| Erlotinib | μ | 1.0 | 9.8 | 17.5 | 3.9 | 638.2 | 1836.9 | 2.5 | 42.2 | 87.5 |
| | σ | 0.04 | 1.3 | 2.0 | 0.1 | 43.3 | 604.2 | 0.5 | 3.2 | 25.3 |
| Gefitinib | μ | 1.0 | 5.2 | 16.3 | 4.1 | 351.9 | 1807.7 | 2.7 | 24.1 | 95.7 |
| | σ | 0.04 | 0.7 | 1.6 | 0.2 | 84.1 | 692.7 | 0.1 | 2.3 | 6.8 |
| Nazartinib | μ | 1.0 | 6.3 | 15.0 | 3.4 | 255.4 | 1514.1 | 2.5 | 25.8 | 53.6 |
| | σ | 0.06 | 0.3 | 1.1 | 0.5 | 61.3 | 217.0 | 0.2 | 1.4 | 5.0 |
| Osimertinib | μ | 1.0 | 2.1 | 12.6 | 3.2 | 38.4 | 836.5 | 2.3 | 13.6 | 64.2 |
| | σ | 0.07 | 0.1 | 1.1 | 0.3 | 5.5 | 136.7 | 0.1 | 0.2 | 23.3 |
| Poziotinib | μ | 1.0 | 14.3 | 15.4 | 4.2 | 716.9 | 1168.4 | 2.8 | 74.1 | 88.4 |
| | σ | 0.07 | 1.1 | 0.9 | 0.4 | 158.4 | 430.8 | 0.2 | 1.1 | 5.3 |
| Rociletinib | μ | 1.0 | 1.3 | 7.0 | 2.9 | 5.1 | 446.1 | 2.4 | 5.3 | 88.0 |
| | σ | 0.10 | 0.1 | 0.6 | 0.2 | 0.9 | 95.1 | 0.2 | 0.1 | 8.0 |
| TAS-6417 | μ | 1.0 | 10.5 | 13.6 | 3.1 | 505.3 | 1235.6 | 2.3 | 50.9 | 71.8 |
| | σ | 0.07 | 1.0 | 1.0 | 0.1 | 100.3 | 312.6 | 0.1 | 7.1 | 4.0 |
| WZ-4002 | μ | 1.0 | 2.6 | 13.5 | 3.8 | 49.8 | 1482.6 | 2.0 | 13.2 | 52.0 |
| | σ | 0.05 | 0.1 | 1.0 | 0.5 | 6.4 | 206.5 | 0.1 | 0.8 | 4.8 |

TABLE 1B

| | | | Low | High | | IPA + | IPA + | | MK-28 + | MK-28 + |
|---|---|---|---|---|---|---|---|---|---|---|
| | | DMSO | EGFRi | EGFRi | IPA | Low EGFRi | High EGFRi | MK-28 | Low EGFRI | High EGFRi |
| Afatinib | μ | 1.0 | 11.7 | 14.7 | 8.7 | 78.8 | 248.5 | 3.9 | 110.8 | 192.0 |
| | σ | 0.07 | 0.8 | 0.9 | 0.7 | 9.3 | 73.5 | 0.8 | 16.1 | 40.8 |
| Dacomitinib | μ | 1.0 | 13.6 | 14.5 | 8.7 | 79.2 | 275.2 | 4.7 | 170.8 | 241.8 |
| | σ | 0.05 | 1.0 | 1.2 | 1.9 | 5.3 | 119.1 | 2.0 | 38.0 | 64.7 |
| Erlotinib | μ | 1.0 | 9.8 | 17.5 | 12.7 | 81.7 | 1748.0 | 7.9 | 250.3 | 447.1 |
| | σ | 0.04 | 1.3 | 2.0 | 1.3 | 24.2 | 195.9 | 7.1 | 6.4 | 44.5 |
| Gefitinib | μ | 1.0 | 5.2 | 16.3 | 13.8 | 34.1 | 576.3 | 5.8 | 104.9 | 465.2 |
| | σ | 0.04 | 0.7 | 1.6 | 1.5 | 7.9 | 88.8 | 2.8 | 14.5 | 50.4 |
| Nazartinib | μ | 1.0 | 6.3 | 15.0 | 10.1 | 44.0 | 205.5 | 6.6 | 130.5 | 421.8 |
| | σ | 0.06 | 0.3 | 1.1 | 0.6 | 6.6 | 54.8 | 4.2 | 13.4 | 43.9 |
| Osimertinib | μ | 1.0 | 2.1 | 12.6 | 8.0 | 9.9 | 67.3 | 4.4 | 72.7 | 220.3 |
| | σ | 0.07 | 0.1 | 1.1 | 0.3 | 2.1 | 9.9 | 1.7 | 15.1 | 20.6 |
| Poziotinib | μ | 1.0 | 14.3 | 15.4 | 10.0 | 107.6 | 132.4 | 5.0 | 181.5 | 295.2 |
| | σ | 0.07 | 1.1 | 0.9 | 1.7 | 28.7 | 35.4 | 2.0 | 1.6 | 28.5 |
| Rociletinib | μ | 1.0 | 1.3 | 7.0 | 11.6 | 6.4 | 142.3 | 6.4 | 49.2 | 268.9 |
| | σ | 0.10 | 0.1 | 0.6 | 0.7 | 0.4 | 42.8 | 3.4 | 16.5 | 39.1 |
| TAS-6417 | μ | 1.0 | 10.5 | 13.6 | 10.3 | 72.3 | 89.5 | 6.8 | 158.3 | 301.6 |
| | σ | 0.07 | 1.0 | 1.0 | 1.8 | 7.5 | 15.0 | 4.6 | 19.0 | 113.1 |
| WZ-4002 | μ | 1.0 | 2.6 | 13.5 | 6.6 | 10.3 | 106.0 | 5.8 | 120.3 | 422.1 |
| | σ | 0.05 | 0.1 | 1.0 | 0.2 | 1.8 | 10.0 | 3.6 | 26.6 | 114.3 |

TABLE 2

| PERK Activator | PERK Activator [ ] (μM) |
|---|---|
| CCT-020312 | 2 |
| DHBDC | 50 |
| IPA | 1 |
| MK-28 | 20 |

TABLE 3

| EGFR Inhibitor | Low [ ] EGFRi (μM) | High [ ] EGFRi (μM) |
|---|---|---|
| Afatinib | 0.01 | 0.1 |
| Dacomitinib | 0.01 | 0.1 |
| Erlotinib | 0.1 | 1 |
| Gefitinib | 0.1 | 1 |
| Nazartinib | 0.1 | 1 |
| Osimertinib | 0.01 | 0.1 |
| Poziotinib | 0.01 | 0.1 |
| Rociletinib | 0.1 | 1 |
| TAS-6417 | 0.01 | 0.1 |
| WZ-4002 | 0.1 | 1 |

### OTHER EMBODIMENTS

This application refers to various issued patent, published patent applications, journal articles, and other publications, each of which is incorporated herein by reference.

The foregoing has been described of certain non-limiting embodiments of the present disclosure. Those of ordinary skill in the art will appreciate that various changes and modifications to this description may be made without departing from the spirit or scope of the present disclosure, as defined in the following claims.

1. A method of treating cancer in an individual in need thereof, comprising administering to the individual a therapeutically effective amount of a combination of (i) a first therapy comprising an EGFR inhibitor, and (ii) a second therapy comprising a PERK activator.

2. The method of claim 1, wherein the PERK activator is selected from the group consisting of 1-(4-((4-((5-cyclopropyl-1H-pyrazol-3-yl)amino)pyrimidin-2-yl)amino)phenyl)-3-(4-(methylthio)phenyl)urea (IPA), 4-[(E)-[(4,6-diphenylpyrimidin-2-yl)-methyl-hydrazono]methyl]benzene-1,2-diol (MK-28), 3-(2,3-dihydrobenzo[b][1,4]dioxin-6-yl)-5,7-dihydroxy-4H-chromen-4-one (DHBDC), 6-bromo-3-[3-(4-bromophenyl)-2-[3-(diethylamino)propanoyl]-3,4-dihydropyrazol-5-yl]-4-phenyl-1H-quinolin-2-one (CCT-020312), and pharmaceutically acceptable salts thereof.

3. The method of claim 1, wherein the EGFR inhibitor is a small molecule EGFR tyrosine kinase inhibitor (EGFR TKI).

4. (canceled)

5. The method of claim 1, wherein the EGFR inhibitor binds reversibly to the EGFR tyrosine kinase domain.

6. The method of claim 1, wherein the EGFR inhibitor binds irreversibly to the EGFR tyrosine kinase domain.

7. The method of claim 1, wherein the EGFR inhibitor is an inhibitor of wild-type EGFR, and/or a mutant form of EGFR, wherein the mutant form of EGFR is exon20insertions, S768I, ex19del, L858R, L861Q, T790M, C797S, C797G, T854A, D761Y, L747S, G719X, L861X, V8343I, V769M, A871E, or any combination thereof.

8. The method of claim 1, wherein the EGFR inhibitor is selected from the group consisting of rac-(E)-N-[4-(3-chloro-4-fluoro-anilino)-7-[rac-(3S)-tetrahydrofuran-3-yl]oxy-quinazolin-6-yl]-4-(dimethylamino)but-2-enamide (afatinib), (E)-N-[4-(3-chloro-4-fluoro-anilino)-7-methoxy-quinazolin-6-yl]-4-(1-piperidyl)but-2-enamide (dacomitinib), N-(3-ethynylphenyl)-6,7-bis(2-methoxyethoxy)quinazolin-4-amine (erlotinib), N-(3-chloro-4-fluoro-phenyl)-7-methoxy-6-(3-morpholinopropoxy)quinazolin-4-amine (gefitinib), N-[7-chloro-1-[rac-(3R)-1-[rac-(E)-4-(dimethylamino)but-2-enoyl]azepan-3-yl]benzimidazol-2-yl]-2-methyl-pyridine-4-carboxamide (nazartinib), N-[2-[2-(dimethylamino)ethyl-methyl-amino]-4-methoxy-5-[[4-(1-methylindol-3-yl)pyrimidin-2-yl]amino]phenyl]prop-2-enamide (osimertinib), 1-[4-[4-(3,4-dichloro-2-fluoro-anilino)-7-methoxy-quinazolin-6-yl]oxy-1-piperidyl]prop-2-en-1-one (poziotinib), N-[3-[[2-[4-(4-acetylpiperazin-1-yl)-2-methoxy-anilino]-5-(trifluoromethyl)pyrimidin-4-yl]amino]phenyl]prop-2-enamide (rocilctinib), N-[rac-(8S)-4-amino-6-methyl-5-(3-quinolyl)-8,9-dihydropyrimido[5,4-b]indolizin-8-yl]prop-2-enamide (TAS-6417), and N-[3-[5-chloro-2-[2-methoxy-4-(4-methylpiperazin-1-yl)anilino]pyrimidin-4-yl]oxyphenyl]prop-2-enamide (WZ-4002), and pharmaceutically acceptable salts thereof.

9. The method of claim 1, wherein the cancer is squamous cell cancer, lung cancer, cancer of the peritoneum, hepatocellular cancer, gastric or stomach cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, B-cell lymphoma, chronic lymphocytic leukemia (CLL), acute lymphoblastic leukemia (ALL), Hairy cell leukemia, chronic myeloblastic leukemia, post-transplant lymphoproliferative disorder (PTLD), abnormal vascular proliferation associated with phakomatoses, edema, or Meigs' syndrome.

10. (canceled)

11. The method of claim 1, wherein the cancer is an EGFR-mutant cancer selected from the group consisting of bladder cancer, glioblastoma, head and neck cancer, breast cancer, cervical cancer, uterine cancer, colorectal cancer, gastroesophageal cancer, non-small cell lung carcinoma (NSCLC), prostate cancer, ovarian cancer, pancreatic cancer, renal cell carcinoma, squamous cell carcinoma, and thyroid cancer.

12. The method of claim 1, wherein the individual is diagnosed with NSCLC, prostate cancer, breast cancer, colon cancer, or pancreatic cancer.

13. The method of claim 12, wherein the NSCLC is metastatic NSCLC or locally advanced NSCLC.

14. The method of claim 12, wherein the individual is diagnosed with EGFR exon 19 deletions positive NSCLC or exon 21 L858R mutations positive NSCLC.

15. The method of claim 12, wherein the individual is diagnosed with EGFR T790M mutation positive NSCLC.

16. (canceled)

17. The method of claim 1, wherein the first and second therapies are administered sequentially.

18. The method of claim 1, wherein the first and second therapies are administered simultaneously.

19. The method of claim 1, wherein the first and second therapies are contained in the same pharmaceutical composition.

20. The method of claim 1, wherein the first and second therapies are contained in separate pharmaceutical compositions.

21. The method of claim 1, wherein the method comprises a third anti-cancer therapy, selected from chemotherapy, radiation therapy, surgery, targeted therapy, immunotherapy, hormone therapy, and stem cell or bone marrow transplant.

22. (canceled)

23. A kit comprising:
(a) a first therapy comprising an EGFR inhibitor;
(b) a second therapy comprising a PERK activator; and
(c) instructions for using the kit in treating cancer in an individual.

24. (canceled)

25. (canceled)

26. (canceled)

27. A composition comprising a unit dosage form of a first therapy comprising an EGFR inhibitor and a unit dosage form of a second therapy comprising a PERK activator.

28-40. (canceled)
